(19) United States
(12) Patent Application Publication
(10) Pub. No.: US 2025/0377430 A1
(43) Pub. Date: Dec. 11, 2025

(54) MEASURING MICROVASCULAR PULSATILITY USING VSASL

(71) Applicant: The Regents of the University of California, Oakland, CA (US)

(72) Inventors: Divya S. Bolar, San Diego, CA (US); Conan Chen, La Jolla, CA (US); Thomas Liu, San Diego, CA (US); Eric C. Wong, Del Mar, CA (US)

(21) Appl. No.: 18/736,413

(22) Filed: Jun. 6, 2024

(51) Int. Cl. G01R 33/563; G01R 33/56; G16H 30/40; G16H 50/30

(52) U.S. Cl. CPC G01R 33/56366; G01R 33/5608; G16H 30/40; G16H 50/30

(57) ABSTRACT

Measuring microvascular pulsatility using velocity-selective ASL. An example method includes: magnetically labelling blood flow in a target area of a subject using a velocity-selective arterial spin labeling (VSASL) technique with a cutoff velocity by performing operations including: applying a first velocity-selective (VS) pulse sequence with the cutoff velocity to mark a leading edge of a blood bolus; and applying a second VS pulse sequence with the cutoff velocity to mark a trailing edge of the blood bolus; acquiring VSASL signals of the blood bolus for voxels corresponding to the target area; for each of the voxels, obtaining signal intensity information over a cardiac cycle by performing retroactive cardiac gating on the acquired VSASL signals corresponding to the voxel; and determining a pulsatility index for the voxel based on the signal intensity information; and generating a voxel-wise pulsatility index map for the target area using the pulsatility indexes of the voxels.

### TECHNICAL FIELD

This document relates to magnetic resonance imaging (MRI).

### BACKGROUND

This document relates to magnetic resonance imaging (MRI). MRI techniques are well known and widely applied in imaging applications across medical, biological, and other fields. For example, arterial spin labeling (ASL) or phase contrast methods based on MRI can be used to reveal information about blood flow within the vasculature of various parts of a subject.

### SUMMARY

Pulsatile blood flow driven by the cardiac cycle has been linked to structural damage to the cerebral microvasculature, and is believed to contribute to dementia, mild cognitive impairment, and other neurovascular diseases. Previous methods have used arterial spin labeling (ASL) or phase contrast to measure pulsatility of cerebral blood volume or flow velocity within the arteries, but few methods exist to measure pulsatility within the microvasculature where the damage is occurring. Velocity-selective ASL (VSASL) is a technique that generates perfusion contrast directly in the microvasculature. The present document presents a theoretical model for CBF pulsatility, applies the model to experimental data acquired in human subjects, and reports in vivo microvascular pulsatility using VSASL.

One aspect of the present document relates to a method for generating a voxel-wise pulsatility index map. An example method includes: magnetically labelling blood flow in a target area of a subject using a velocity-selective arterial spin labeling (VSASL) technique with a cutoff velocity by performing operations including: applying a first velocity-selective (VS) pulse sequence with the cutoff velocity to mark a leading edge of a blood bolus; and applying a second VS pulse sequence with the cutoff velocity to mark a trailing edge of the blood bolus; acquiring VSASL signals of the blood bolus for voxels corresponding to the target area; for each of the voxels, obtaining signal intensity information over a cardiac cycle by performing retroactive cardiac gating on the acquired VSASL signals corresponding to the voxel; and determining a pulsatility index for the voxel based on the signal intensity information; and generating a voxel-wise pulsatility index map for the target area using the pulsatility indexes of the voxels.

Another exemplary aspect relates to an MRI system for performing the above-described methods described in this document. A further exemplary aspect relates to processor-executable code and stored in one or more non-transitory computer-readable storage media. The code included in the computer readable storage media when executed by one or more processors, causes the one or more processors to implement the methods described in this document.

The above and other aspects and their implementations are described in greater detail in the drawings, the descriptions, and the claims.

### BRIEF DESCRIPTION OF THE DRAWINGS

FIG. 1 illustrates a network of vasculature in the brain of a subject.

FIG. 2 illustrates a VSASL labeling process according to some embodiments of the present document.

FIG. 3 shows a block diagram illustrating an overview of an environment 300 in which some implementations of the disclosed technology can operate according to some embodiments of the present document.

FIG. 4 shows a schematic of an MRI system configured according to some embodiments of the present document.

FIG. 5 is a block diagram illustrating an overview of devices on which some implementations can operate.

FIG. 6 illustrates a flowchart of a process for generating a pulsatility index map according to some embodiments of the present document.

FIG. 7 illustrates graphical representation of computed optimal tau τₒₚₜ.

FIG. 8 illustrates fits of the sinc model to the PI values measured from the τ-stepping protocol according to some embodiments of the present document.

FIG. 9 show the demeaned and normalized perfusion-weighted image (PWI) signal vs ϕ/2π for a select slice of a subject according to some embodiments of the present document.

FIG. 10 shows results demonstrating repeatability of PI(τ=500 ms) across subjects according to some embodiments of the present document.

FIG. 11 are a plot of PI(τ=Γ/2) vs age according to some embodiments of the present document.

FIG. 12 is a voxel-wise PI map determined according to some embodiments of the present document

FIG. S1 shows results of fitting τ-stepping data to the sinc model according to some embodiments of the present document.

FIG. S2a though S2d show the results of τ-stepping analysis comparison according to some embodiments of the present document.

FIG. S3 illustrates the regimes of τ according to some embodiments of the present document.

FIG. S4 illustrates noise simulations for PI according to some embodiments of the present document.

FIG. S5 are the SNR curves shown in diagram (c) of FIG. S4.

FIG. S6 is an illustration of the 2nd-order Fourier models in the iterative denoising algorithm according to some embodiments of the present document.

FIG. S7 shows PIs values plotted vs t and fitting results according to some embodiments of the present document.

FIG. S8 shows results of the repeatability experiment according to some embodiments of the present document.

FIG. S9 illustrates retroactive cardiac gating according to some embodiments of the present document.

Like reference symbols and designations in the various drawings indicate like elements.

### DETAILED DESCRIPTION

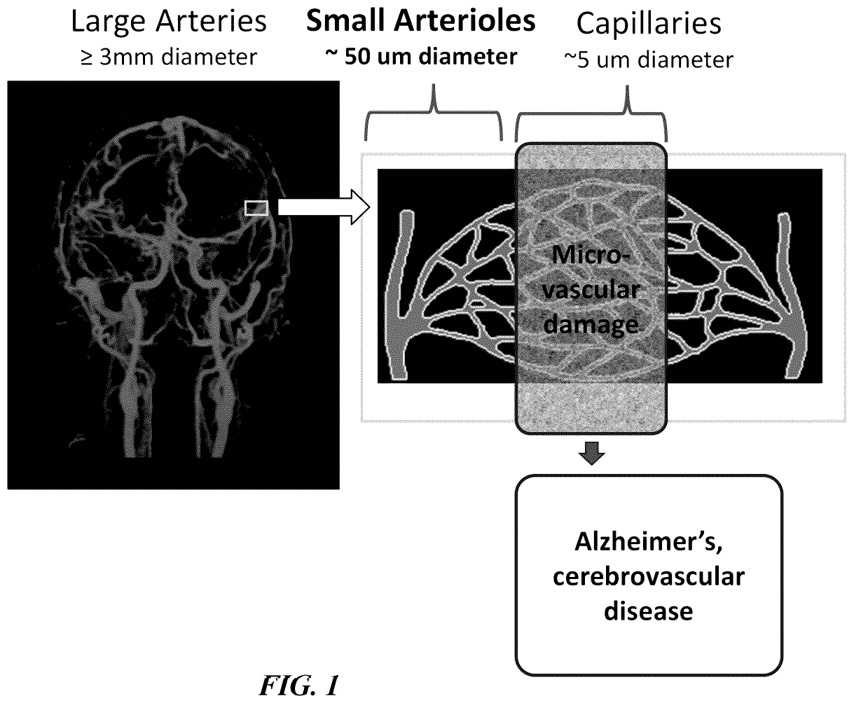

Pulsatile blood flow driven by the cardiac cycle has been linked to structural damage in the cerebral microvasculature and cognitive disorders such as mild cognitive impairment, Alzheimer's disease, and other dementias. As illustrated in FIG. 1, large arteries (e.g., arteries whose diameter is at least three millimeters) may deliver pulsatile blood flow to the brain via a network of small arterioles (e.g., whose diameter is approximately 50 micrometers) and capillaries whose diameter is approximately 5 micrometers). In healthy subjects, this flow pulsatility is dampened by compliant arteries as the pulse wave travels toward the distal microvasculature and brain parenchyma. However, if this dampening is insufficient, for example, due to pathologic changes in the vasculature, then excess pulsatile energy may reach the smaller vessels, where the resulting exposure and subsequent damage may be a precursor to these aforementioned or other cognitive disorders. There has been work assessing the pulsatility and vessel wall compliance at the large cerebral arteries. However, techniques measuring pulsatility in the microvasculature itself, the site where damage may primarily occur, are lacking. Such techniques may help clarify the mechanistic links between microvascular pulsatility, tissue damage, and eventual neurodegeneration, which are not yet fully understood. Furthermore, since neurovascular factors such as flow pulsatility are viewed as early and modifiable risk factors contributing to these disorders, the ability to measure these biomarkers may help develop strategies for early detection and intervention.

Historically, microvascular pulsatility has been challenging to measure due to the very small size of microvascular vessels and their relatively slow flow. Recent technical advances in MRI have enabled pulsatility measurements in small perforating arteries using phase-contrast MRI by leveraging an ultra-high field strength of 7 T to resolve and measure such individual vessels. Additional advances to data acquisition (higher temporal resolution, dual velocity encoding) and post-processing (automated vessel detection) have improved the usability and robustness of the technique even further. However, this approach remains challenging at the lower field strengths common on clinical scanners, which limits its potential for clinical translation. The present document describes an alternative approach, a cerebral perfusion technique called velocity-selective arterial spin labeling (VSASL) to measure microvascular pulsatility, which can be performed on clinical 3T scanners with a simple whole-brain scan prescription and can generate cerebral microvascular pulsatility maps on a voxel-wise basis.

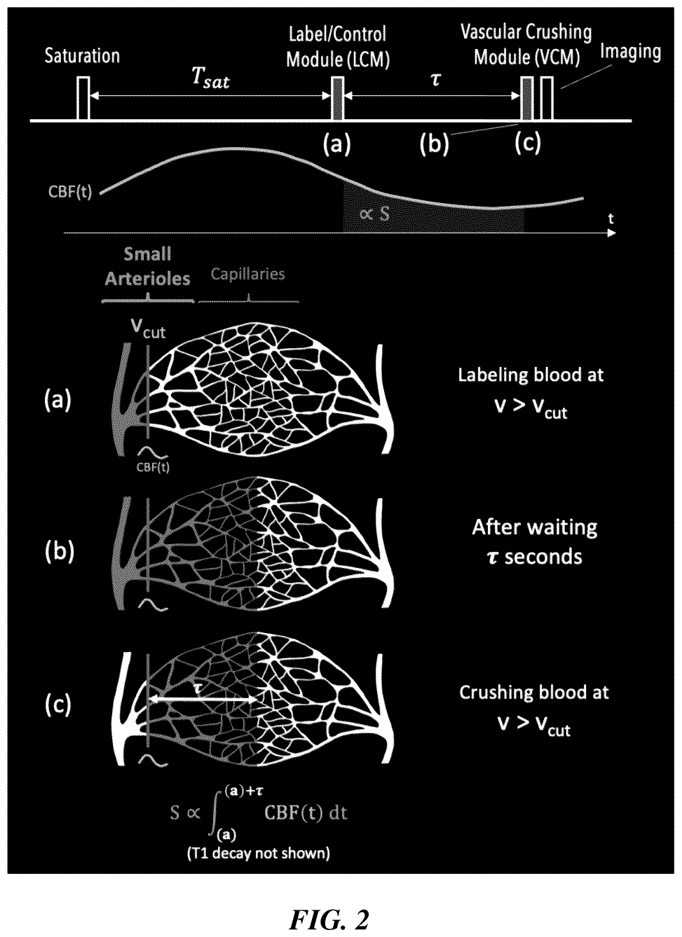

VSASL is a specific variant of arterial spin labeling (ASL), a family of methods used to measure perfusion by magnetically labeling a bolus of arterial blood, allowing it to flow into the microvasculature or tissue of interest, and then acquiring images. While the traditional ASL variants generate the magnetic label within the feeding extracranial carotid and vertebral arteries, VSASL may generate the label in smaller, more distal arteries. In VSASL, a user-specified sequence parameter of cutoff velocity (ν_{cut}) may determine or affect the location along the vasculature where the leading and trailing edges of the bolus are defined, as illustrated in FIG. 2. Using a typical setting of νᵥᵤₜ=2 cm/s, the sequence may define the labeled bolus in small arterioles with vessel diameters of about 50 micrometers, which may be referred to as the microvasculature. In the VSASL sequence as disclosed herein, a first velocity-selective (VS) pulse sequence (e.g., a label/control module (LCM)) may label blood flowing faster than ν_{cut}, thus defining or marking the leading edge of the labeled bolus (Diagram (a) of FIG. 2). This may be followed by a delay corresponding to a bolus duration τ (a user-specified sequence parameter), during which the bolus flows distally toward its target tissue while decelerating below ν_{cut} in the process (Diagraph (b) of FIG. 2). A second VS pulse sequence (e.g., a vascular crushing module (VCM)) may then be applied to label (e.g., by saturating) remaining labeled blood still flowing faster than ν_{cut}, thus defining or marking the trailing edge of the labeled bolus (Diagram (c) of FIG. 2). The labeled bolus signal may be proportional to the volume of blood that flows across the ν_{cut} boundary in the time between the first VS pulse sequence (e.g., LCM) and the second VS pulse sequence (e.g., VCM), thus making the labeled bolus signal (i.e., VSASL signal) sensitive to or indicative of the flow rate and its variation across the cardiac cycle.

Previous work using a single VS labeling module (the LCM) measured fluctuations of up to 36% in the amount of arterial label generated. This measurement was made in an arterial region of interest (ROI) and was primarily weighted by blood volume (as opposed to blood flow) since only the LCM was applied without an accompanying VCM. The present document discloses a standard VSASL sequence design that includes a second VS pulse sequence (e.g., VCM) to achieve blood flow weighting, and leverages the microvascular specificity of VSASL along with retrospective cardiac gating to measure blood flow pulsatility in the microvasculature.

The present document describes a theory relating pulsatile blood flow to the pulsatility of retrospectively-gated VSASL signal, and a model describing the dependence of the microvascular pulsatility measurement on bolus duration τ. This model may be used to determine a theoretically optimal τ value that may improve or maximize the SNR of the pulsatility measurements. The present document describes validation of the predicted τ dependence of the pulsatility measurement and assessment of its intrasession test-retest repeatability using experimental VSASL data acquired in human subjects with a broad range of ages and heart rates. The present document also illustrates the association between pulsatility and age and demonstrate the feasibility of a novel voxel-wise pulsatility mapping approach that may facilitate regional pulsatility measurements in various applications.

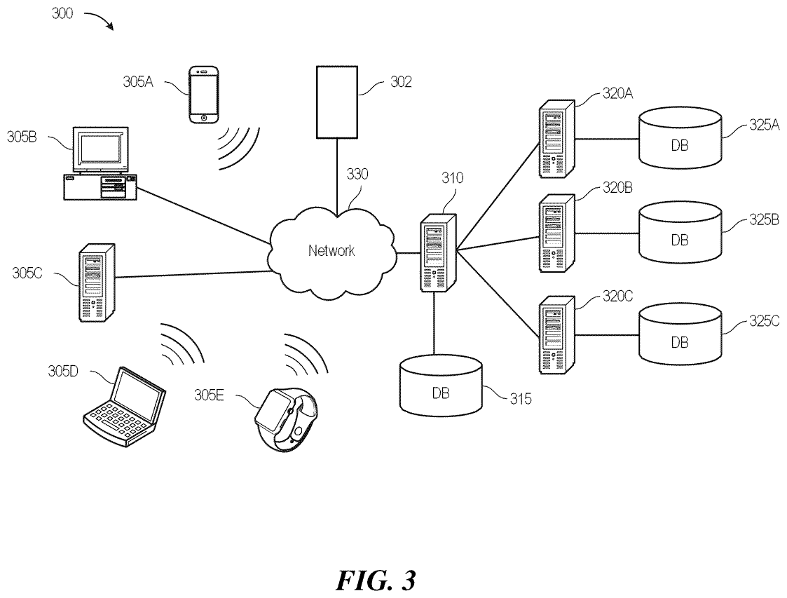

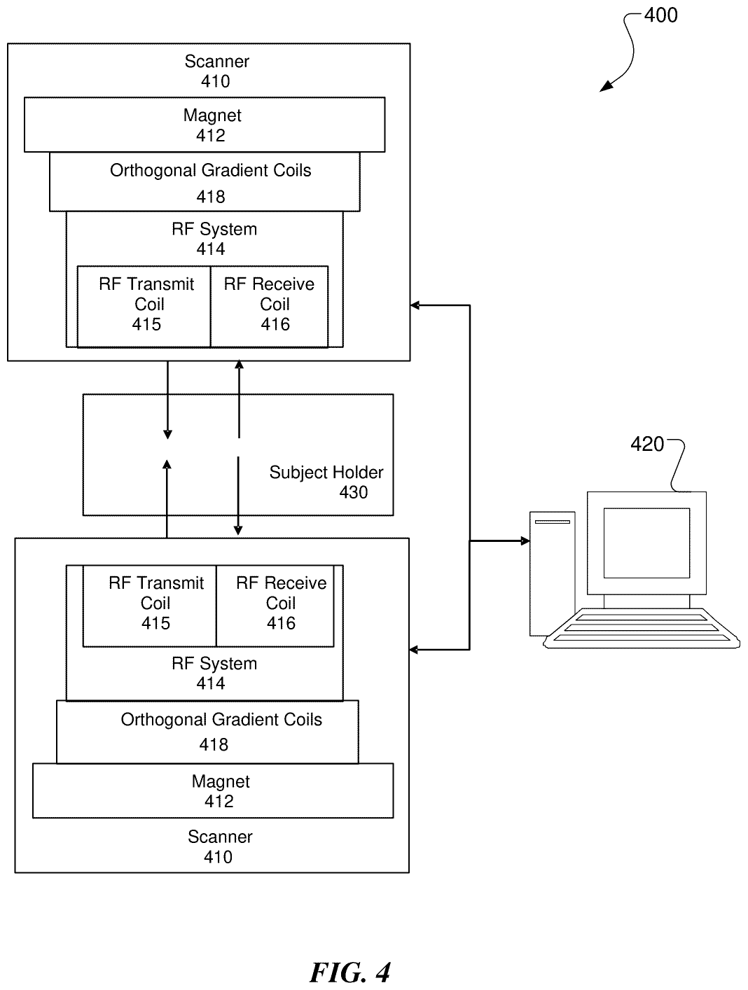

FIG. 3 is a block diagram illustrating an overview of an environment 300 in which some implementations of the disclosed technology can operate. Environment 300 can include one or more client computing devices 305A-E (collectively referred to as “client computing devices 305”) and an MRI system 302. Client computing devices 305 and the MRI system 302 (e.g., system 400 as illustrated in FIG. 4) can operate in a networked environment using logical connections through network 330 to one or more remote computers, such as a server computing device.

The client computing device 305 may be configured to enable a user interaction between a user and the MRI system 302. For example, the client computing device 305 may receive an instruction to cause the MRI system 302 to scan a subject (e.g., a patient), or a portion thereof, from the user (e.g., a physician, an imaging technician, a healthcare provider, a researcher, etc.). As another example, the client computing device 305 may receive signals acquired by the MRI system 302, and/or a processing result (e.g., an image, a map of a physiological parameter (e.g., pulsatility indexes as described herein) of the subject) from the MRI system 302 or another computing device and display the processing result to the user. In some embodiments, the client computing device 305 may include a mobile device 305A, a desktop computer 305B, a server 305C, a tablet computer 305D, a smartwatch 306E, or the like, or a combination thereof. For example, the mobile device 305A may include a mobile phone, a personal digital assistant (PDA), or the like, or a combination thereof. In some embodiments, the client computing device 305 may include an input device, an output device, etc. The input device may include alphanumeric and other keys that may be input via a keyboard, a touch screen (for example, with haptics or tactile feedback), a speech input, an eye tracking input, a brain monitoring system, or any other comparable input mechanism. The input information received through the input device may be transmitted to the server 310, the MRI system 302, the storage device 315 or 325, etc., via, for example, a bus, for further processing. Other types of the input device may include a cursor control device, such as a mouse, a trackball, or cursor direction keys, etc. The output device may include a display, a speaker, a printer, or the like, or a combination thereof.

In some implementations, server 310 can be an edge server which receives client requests and coordinates fulfillment of those requests through other servers, such as servers 320A-C. Server computing devices 310 and 320 can include computing systems, such as a data processing apparatus 420 of the MRI system 400 as illustrated in FIG. 4. Though each server computing device 310 and 320 is displayed logically as a single server, server computing devices can each be a distributed computing environment encompassing multiple computing devices located at the same or at geographically disparate physical locations. In some implementations, each server 320 corresponds to a group of servers.

Client computing devices 305 and server computing devices 310 and 320 can each act as a server or client to other server/client devices. Server 310 can connect to a database 315. Servers 320A-C can each connect to a corresponding database 325A-C. As discussed above, each server 320 can correspond to a group of servers, and each of these servers can share a database or can have their own database. Databases 315 and 325 can warehouse (e.g., store) information such as table data, column data, value filter data, user interface data, database element data, selection data, root table data, code snippet data, join query data, query template data, connection data. Though databases 315 and 325 are displayed logically as single units, databases 315 and 325 can each be a distributed computing environment encompassing multiple computing devices, can be located within their corresponding server, or can be located at the same or at geographically disparate physical locations.

Network 330 can be a local area network (LAN) or a wide area network (WAN), but can also be other wired or wireless networks. Network 330 may be the Internet, a mobile phone network, a mobile voice or data network (e.g., a 5G or long term evolution (LTE) network), a cable network, a public switched telephone network, a short-range wireless communication network (e.g., Bluetooth or Near Field Communications (NFC)), or some other public or private network. Client computing devices 305 can be connected to network 330 through a wired or wireless network interface, such as a satellite path, a fiber-optic path, a cable path, a path that supports internet communications (e.g., internet protocol television (IPTV)), free-space connections (e.g., for broadcast or other wireless signals), etc. While the connections between server 310 and servers 320 are shown as separate connections, these connections can be any kind of local, wide area, wired, or wireless network, including network 330 or a separate public or private network. As described in further detail herein, the client computing devices 305 and the MRI system 302 can operate according to an edge computing protocol (e.g., an edge computing decryption protocol).

FIG. 4 shows a schematic of an MRI system 400 configured according to some embodiments of the present document. The MRI system 400 may be an exemplary implementation of the MRI system 302 as illustrated in FIG. 3. Techniques as disclosed in this document can be implemented using the MRI system 400. The MRI system 400 can be implemented using any one of various MRI scanners such as a 1.5 Tesla Signa TwinSpeed scanner (available from GE Healthcare Technologies, Milwaukee, WI.), a Siemens Prisma 3.0 Tesla system, etc. The MRI system 400 may include a scanner 410, a data processing apparatus 420, and a subject holder or table 430 for holding a subject. The scanner 410 may include a main magnet 412, three orthogonal gradient coils 418 and a radio frequency (RF) system 414. The main magnet 412 may be configured to provide a constant, homogeneous magnetic field. The three orthogonal gradient coils 418 may be configured to provide three orthogonal, controller magnetic gradients used to acquire image data of a desired slice by generating an encoded and slice-selective magnetic field. The RF system 414 includes an RF transmit coil 415 and an RF receive coil 416 configured to transmit and receive RF pulses. The RF system 414 can further include an RF synthesizer (not shown) and a power amplifier (not shown). In some implementations, an integrated transceiver coil (not shown) can be implemented instead of the separate transmit coil 415 and receive coil 416 for transmitting and receiving RF signals. For example, a close-fitting smaller coil can improve image quality when a small region is being imaged. Further, various types of coils that are placed around specific parts of a body (e.g., the head, knee, wrist, etc.) or even internally can be implemented depending on the sample and imaging applications.

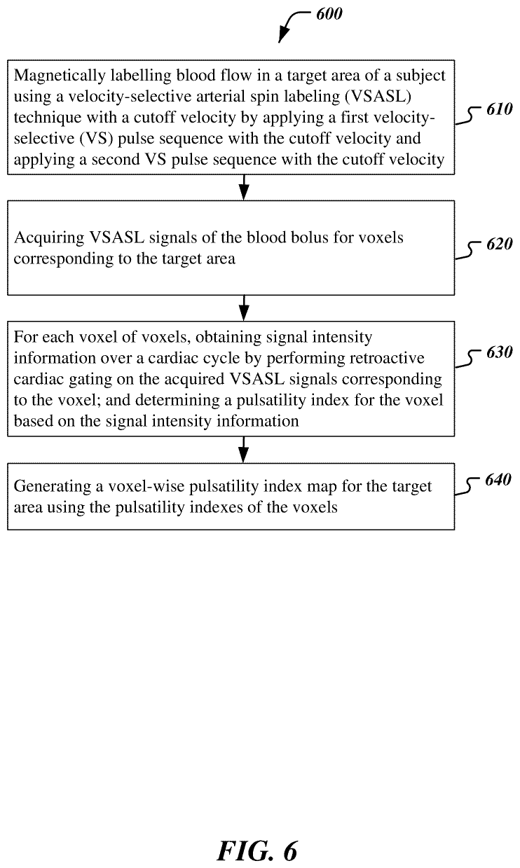

The MRI system 400 may be configured to perform the techniques disclosed in this document. In particular, the MRI system 400 may be configured to implement the methods described elsewhere in the present document, e.g., the process 600 as illustrated in FIG. 6. The RF system 414 may be configured to apply to a subject (e.g., a patient), or a portion thereof (e.g., a target area of a subject) a non-selective inversion RF pulse, a slice-selective inversion RF pulse, and a half RF excitation pulse. The three orthogonal coils 418 may be configured to apply slice-selective magnetic field gradients (of a first polarity and a second polarity) and magnetic readout gradients. The data processing apparatus (e.g., a computer) 420 may be configured to receive and process the acquired data to obtain desired images or information relating to pulsatility of a target area of a subject.

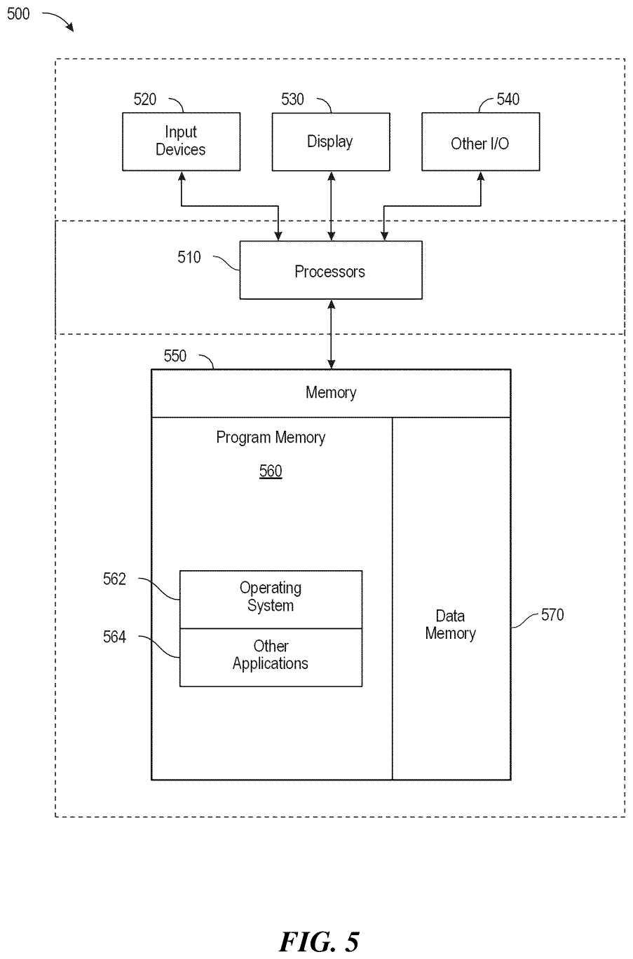

FIG. 5 is a block diagram illustrating an overview of devices on which some implementations can operate. The devices can include hardware components of a device 500 that illustrates an exemplary implementation of the data processing device 420 as illustrated in FIG. 4. Device 500 can include one or more input devices 520 that provide input to the Processor(s) 510 (e.g., CPU(s), GPU(s), HPU(s), etc.), notifying it of actions. The actions can be mediated by a hardware controller or control circuit that interprets the signals received from the input device and communicates the information to the processors 510 using a communication protocol. Input devices 520 include, for example, a mouse, a keyboard, a touchscreen, an infrared sensor, a touchpad, a wearable input device, a camera- or image-based input device, a microphone, or other user input devices.

Processors 510 can be a single processing unit or multiple processing units in a device or distributed across multiple devices. Processors 510 can be coupled to other hardware devices, for example, with the use of a bus, such as a peripheral component interconnect (PCI) bus or small computer system interface (SCSI) bus. The processors 510 can communicate with a hardware controller or control circuit for devices, such as for a display 530. Display 530 can be used to display text and graphics. In some implementations, display 530 provides graphical and textual visual feedback to a user. In some implementations, display 530 includes the input device as part of the display, such as when the input device is a touchscreen or is equipped with an eye direction monitoring system. In some implementations, the display is separate from the input device. Examples of display devices include: a liquid crystal display (LCD) screen, a light emitting diode (LED) screen, a projected, holographic, or augmented reality display (such as a heads-up display device or a head-mounted device), and so on. Other input/output (I/O) devices 540 can also be coupled to the processor, such as a network card, video card, audio card, USB, firewire or other external device, camera, printer, speakers, compact disc read-only memory (CD-ROM) drive, digital versatile disc (DVD) drive, disk drive, or Blu-Ray device.

In some implementations, the device 500 also includes a communication device capable of communicating wirelessly or wire-based with a network node. The communication device can communicate with another device or a server through a network using, for example, transmission control protocol/internet protocol (TCP/IP) protocols. The device 500 can utilize the communication device to distribute operations across multiple network devices.

The processors 510 can have access to memory 550 in a device or distributed across multiple devices. Memory 550 may include one or more of various hardware devices for volatile and non-volatile storage, and can include both read-only and writable memory. For example, memory 550 can include random access memory (RAM), various caches, CPU registers, read-only memory (ROM), and writable non-volatile memory, such as flash memory, hard drives, floppy disks, CDs, DVDs, magnetic storage devices, tape drives, and so forth. Memory 550 is not a propagating signal divorced from underlying hardware; memory 550 is thus non-transitory. Memory 550 can include program memory 560 that stores programs and software, such as an operating system 562 and other application programs 564. Memory 550 can also include data memory 570, e.g., table data, column data, value filter data, user interface data, database element data, selection data, root table data, code snippet data, join query data, query template data, connection data, configuration data, settings, user options or preferences, etc., which can be provided to the program memory 560 or any element of the device 500.

Some implementations can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, gaming consoles, tablet devices, multiprocessor systems, microprocessor-based systems, set-top boxes, programmable consumer electronics, network PCs, minicomputers, mainframe computers, distributed computing environments that include any of the above systems or devices, or the like.

FIG. 6 illustrates a flowchart of a process 600 for generating a pulsatility index map according to some embodiments of the present document. The process 600 may start at block 610 by magnetically labelling blood flow in a target area of a subject using a VSASL technique with a cutoff velocity (ν_{cut}). The magnetic labelling may include applying a first VS pulse sequence with the cutoff velocity to mark a leading edge of a blood bolus and applying a second VS pulse sequence with the cutoff velocity to mark a trailing edge of the blood bolus. At least one of the first VS pulse sequence or the second VS pulse sequence may include a velocity-selective saturation (VSS) pulse sequence, a VS inversion (VSI) pulse sequence, etc. The first VS pulse sequence or the second VS pulse sequence may include pulse sequences of a same type, or different types. Merely by way of example, the second VS pulse sequence may include a VSS pulse sequence (e.g., an eight-segment B₀/B₁⁺ insensitive rotation (BIR-8) train), while the first VS pulse may include a different type of pulse sequence (e.g., a VSI pulse sequence). The first VS pulse sequence may constitute the LCM. The second VS pulse sequence may constitute VCM. Additional description regarding the LCM and VCM may be found elsewhere in the present document. Because blood signals flowing faster than the cutoff velocity ν_{cut} is labeled by the first VS pulse sequence (e.g., LCM) and the second VS pulse sequence (e.g., VCM), signals from the slow or substantially stagnant molecules, such as those in the tissue surrounding the blood vessels in the target area may be negligible. Accordingly, the velocity selective ASL may obviate the need to delineate the blood vessels through methods such as image segmentation in subsequent data analysis.

The application of the second VS pulse sequence may be separated from the application of the first VS pulse sequence by a bolus duration. In some embodiments, the process 600 may include selecting the bolus duration based on a cardiac period of the subject. In some embodiments, the bolus duration may be (approximately) half of the cardiac period of the subject. In some embodiments, the process 600 may further include applying one or more background suppression (BGS) pulses between the first VS pulse sequence and the second VS pulse sequence.

The cutoff velocity may determine or affect the location along the vasculature where the leading and trailing edges of the bolus are defined, as illustrated in FIG. 2. For example, the cutoff velocity of 2 centimeters per second corresponds to the VSASL bolus in the microvascular regime. The value of the cutoff velocity can be adjusted to target other segments of the arterial network. Increasing the cutoff velocity may shift the VSASL bolus-defining location more upstream into larger vessels, whereas decreasing the cutoff velocity may shift the labeling region further distally toward the capillaries. In some embodiments, the process 600 may include selecting the cutoff velocity based on a dimension of blood vessels in the target area or a location of the target area along an arterial network of the subject. Varying the cutoff velocity may be useful for evaluating pulsatility along the arterial tree using a single approach and assessing metrics like dampening factor.

At block 620, the process 600 may continue by acquiring VSASL signals of the blood bolus for voxels corresponding to the target area. The VSASL signal may be acquired by performing a VSASL scan of the target area. The scans may follow the application of the second VS pulse sequence by a post-labelling delay (PLD). PLD may refer to the time between the second VS pulse sequence and the image acquisition. In some embodiments, PLD in VSASL may be set to a minimal value to reduce the T1 decay. Merely by way of example, the target area is the brain of the subject, and the PLD may be set to a minimum needed to accommodate one or more modules (e.g., one or more spectrally-selective fat-saturation module, and/or one or more inferior saturation modules, etc.) to reduce or minimize CSF inflow effects prior to readout. In some embodiments, raw VSASL signals acquired during the VSASL scan may undergo co-registration and/or motion correction to reduce or eliminate artifacts. Additional pre-processing steps may include denoising, masking, normalization, smoothing, spatial filtering, temporal filtering, or the like, or a combination thereof.

At block 630, the process 600 may continue by determining pulsatility index for each of a plurality of voxels. The process 600 may include for each voxel of the voxels corresponding to the target area, obtaining signal intensity information over a cardiac cycle by performing retroactive cardiac gating on the acquired VSASL signals corresponding to the voxel; and determining a pulsatility index for the voxel based on the signal intensity information. In some embodiments, the VSASL scan may last over multiple cardiac cycles. For a voxel, individual VSASL signals may be from one of the multiple cardiac cycles. The process 600 may perform retroactive cardiac gating by assigning a cardiac phase to each of the VSASL signals. See also, e.g., section S9. Based on signal intensity information of the voxel corresponding to the gated VSASL signals, the process 600 may determine the pulsatility index of the voxel. For example, the pulsatility index may be determined using the extremum (e.g., maximum and minimum signal intensities) and the mean signal intensity of the voxel's signal intensity information. As an illustration, the pulsatility index of a voxel may be the ratio of the difference between the extremum signal intensities (the range between maximum and minimum signal intensities) to the mean signal intensity.

At block 640, the process 600 may continue by generating a voxel-wise pulsatility index map for the target area using the pulsatility indexes of the voxels. The map may be three-dimensional. The map may be further processed to provide a two-dimensional map. For example, a two-dimensional map for a slice of tissue within the target area may be obtained by specifying the location information of the slice in the target area. As another example, a two-dimensional map representing the projection of signal intensities onto a specific plane within the three-dimensional map may be obtained.

Additional details regarding the technique are provided below with reference to cerebral microvasculature. However, it is understood that this is for illustration purposes without being limiting. The technology may be applied in examining vasculature of different dimensions (e.g., by setting different cutoff velocities) and/or in other areas of a subject. For example, the technique may be applied to examine target areas including a lung, a kidney, or the liver of the subject.

Section headings are used only to improve readability of the disclosed subject matter. The section headings do not in any way limit the scope of the disclosed and claimed subject matter.

### A. Theory

### A.1 Pulsatility of VSASL Signal

The control-label subtraction signal (denoted S) from a VSASL scan represents the signal of the labeled blood being delivered to the microvasculature. This signal S is typically modeled as being proportional to CBF₀·τ·exp(−τ/T_{1b})), where CBF₀ reflects uniform (non-pulsatile) cerebral blood flow, τ is bolus duration, and exp(−τ/T_{1b}) is the T₁ decay weighting factor (where T_{1b} is the T₁ of blood).

In the case of time-varying, pulsatile blood flow, the product CBF₀·τ becomes an integration of CBF(t) over the duration of the bolus, and the continuous-time VSASL signal S(t) can be described as:

$$S ( t ) ∝ CBF ( t ) * rect ( \frac{t}{τ} ) exp ( - \frac{τ}{{T}_{1 b}} ) , \qquad ( 1 )$$

where * denotes convolution and rect(t/τ) is a rectangular function of width τ representing the bolus. To examine cardiac-driven pulsatility, CBF(t) may be modelled with a simple 2nd-order Fourier model, which has previously been used for cardiac-gated measurements in ASL. This model of CBF(t) is:

$$CBF ( t ) = {b}_{0} + {\sum }_{k = 1}^{2} [ {b}_{cos , k} \cdot cos ( \frac{2 π kt}{Γ} ) + {b}_{sin , k} \cdot sin ( \frac{2 π kt}{Γ} ) ] , \qquad ( 2 )$$

where b₀=CBF₀, b_{cos,1}, b_{sin,1}, b_{cos,2} and b_{sin,2} are the Fourier coefficients and I represents the cardiac period (RR interval, or inverse heart rate). Evaluating the convolution in Eq. 1 yields:

$$\begin{matrix}S ( t ) ∝ {b}_{0} \cdot τ exp ( - \frac{τ}{{T}_{1 b}} ) + {\sum }_{k = 1}^{2} [ {b}_{cos , k} \cdot τ exp ( - \frac{τ}{{T}_{1 b}} ) sinc ( \frac{k τ}{Γ} ) cos ( \frac{2 π kt}{Γ} ) … \\ + {b}_{sin , k} \cdot τ exp ( - \frac{τ}{{T}_{1 b}} ) sinc ( \frac{k τ}{Γ} ) sin ( \frac{2 π kt}{Γ} ) ] ,\end{matrix} \qquad ( 3 )$$

a smoothed version of the CBF(t) driving function with the coefficients modified by an order-dependent sinc (kτ/Γ) term, where

$$sinc ( x ) = \frac{sin ( π x )}{π x} .$$

The pulsatility of a given waveform S can be quantified via the pulsatility index (PI), which is given by:

$$PI = \frac{{S}_{max} - {S}_{min}}{{S}_{mean}} , \qquad ( 4 )$$

where Sₘₐₓ is the maximum of S(t), Sₘᵢₙ is the minimum, and Sₘₑₐₙ is the mean.

In physiological flow waveforms, the fundamental (1st-order) frequency is typically the largest harmonic in the power spectrum. By assuming

$$\sqrt{{b}_{cos , 1}^{2} + {b}_{sin , 1}^{2}} ▯ \sqrt{{b}_{cos , 2}^{2} + {b}_{sin , 2}^{2}}$$

and neglecting the 2nd-order terms of S(t) (see Section S1 for supplementary details on this approximation), Eq. 4 can then be applied to the VSASL signal in Eq. 3 to yield:

$$PI ( τ ) = A \cdot \vert sinc ( \frac{τ}{Γ} ) \vert , \qquad ( 5 )$$

where

$$A = \frac{2 \sqrt{{b}_{cos , 1}^{2} + {b}_{sin , 1}^{2}}}{{b}_{0}}$$

is a lumped fitting parameter. This simple expression indicates that PI exhibits a sinc-shaped dependence on the ratio of the bolus duration τ (an adjustable VSASL scan parameter) to cardiac period Γ. Note that this form predicts that PI will vanish when σ=Γ, a feature that will be examined later with in vivo data.

### A.2 Optimal Choice of Tau

The value of τ that maximizes the SNR of PI may be determined. In Section S3, the SNR of the PI measurement is shown to be approximately proportional to the product of the SNR of the VSASL signal (∝τ·exp(−τ/T_{1b}) and the magnitude of PI (Eq. 5):

$$SNR ( τ ) = τ \cdot exp ( - \frac{τ}{{T}_{1 b}} ) \cdot \vert sinc ( \frac{τ}{Γ} ) \vert \qquad ( 6 )$$

This expression may be maximized at τₒₚₜ:

$${τ}_{opt} = Γ \cdot ( \frac{1}{2} - \frac{1}{π} \cdot arctan ( \frac{Γ}{π \cdot {T}_{1 b}} ) ) , \qquad ( 7 )$$

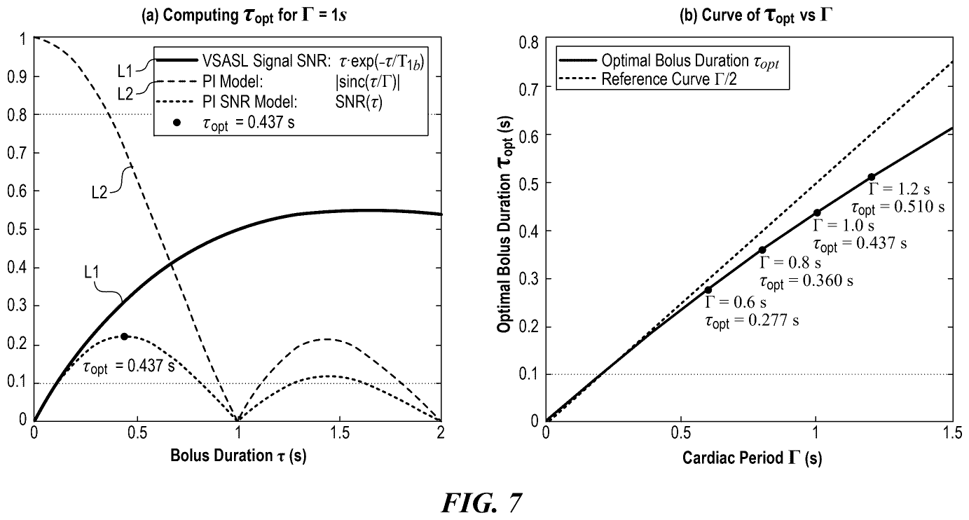

yielding an optimal value slightly below Γ/2. Diagram (a) of FIG. 7 is a graphical demonstration of optimizing Eq. 6 to obtain Eq. 7. For an example cardiac period of Γ=1 s, the optimal tau is computed as τₒₚₜ=0.437 s and indicated on the graph. Diagram (b) of FIG. 7 then plots Eq. 7 for τₒₚₜ over a range of cardiac periods. The discrepancy between SNR (τₒₚₜ) and SNR (Γ/2) is quite small (<3% for Γ in [0.6, 1.2] s), suggesting that choosing Γ=τ/2 is a reasonable rule of thumb for selecting a bolus duration τ that maximizes SNR.

A supplementary analysis on optimal τ is provided in Section S3, which covers the theoretical derivation of Eq. 6 and discuss PI measurement bias (which is negligible in the regime of τₒₚₜ). Monte Carlo simulations with measurement noise are also performed to numerically compute the SNR of PI as a function of t, with FIG. S4 showing that τₒₚₜ indeed yields close to the maximum SNR of the PI measurement.

### B. Methods

### B.1 Overview

A primary cohort of 7 healthy subjects (3 females and 4 males, aged 38.1±14.9 years), plus a secondary cohort of 10 relatively older healthy subjects (9 females and 1 male, aged 65.9±10.0 years), were enrolled in this study. The study was approved by the UCSD Institutional Review Board (IRB), and informed consent was obtained from all participants. The experiment includes a series of MRI scans, all acquired on a 3T MAGNETOM Prisma (Siemens, Erlangen, Germany) using a 64ch head/neck receiver coil. During the MRI scans, photoplethysmography (PPG) data were collected from the subject's index finger for retrospective gating.

### B.2 MRI Acquisition

The scanning protocol for the primary cohort included a localizer, a T1 structural scan, and then a series of six VSASL scans: two at τ=500 ms (to evaluate test-retest repeatability near τₒₚₜ per Eq. 7), and then one each at progressively longer values (750/1000/1250/1500 ms) for a τ-stepping experiment to test for the predicted sinc-dependence described in Eq. 5. All primary cohort subjects completed the full protocol, with a few exceptions noted in Table 2. The secondary cohort of 10 subjects underwent a short scanning protocol includes a T1 structural scan and a single VSASL scan at τ=500 ms, with all subjects completing this short protocol. T1 structural scan parameters are provided in Section S4.

The VSASL pulse sequence included a velocity-selective preparation module and an accelerated 3D gradient- and spin-echo (GRASE) readout. VSASL scans were configured for the five different bolus durations of τ=500/750/1000/1250/1500 ms, with repetition time (TR) adjusted to accommodate the τ setting of a given scan. Other sequence timing parameters τₛₐₜ and post-labeling delay (PLD) were kept fixed across all scans at τₛₐₜ=1500 ms and PLD=100 ms, respectively. The PLD was kept to the minimum needed to accommodate two spectrally-selective fat-saturation modules and two inferior saturation modules (to minimize CSF inflow effects) immediately prior to readout. Two background suppression (BGS) pulses were inserted for each scan between the LCM and the VCM. Additional sequence timing details are provided in Table 1.

The 3D GRASE readout was configured as: matrix size=56×56×24; voxel size=4×4×6 mm³; FOV=224×224×144 mm³; 10% Slice Oversampling; 6/8 Phase Partial Fourier; 2×2 GRAPPA Acceleration; Turbo Factor=13; EPI Factor=21; 1 segment (single-shot acquisition); flip angle=120 degrees; TE=12.62 ms.

An eight-segment B₀/B₁⁺ insensitive rotation (BIR-8) train was used for both the LCM and the VCM, with velocity weighting in the inferior-superior direction. The BIR-8 train was configured with a cutoff velocity of ν_{cut}=2 cm/s, with the cutoff velocity defined as the first zero-crossing of the laminar flow response of the label condition.

### B.3 Data Pre-Processing

For each subject, all VSASL scans were co-registered and motion-corrected using AFNI's 3 dvolreg command. The Tl structural scans were processed using FSL's fsl_anat command, producing a whole-brain mask and a partial volume map of gray matter (GM). The GM partial volume map was thresholded at 0.8 (80%) to produce a GM mask. The brain and GM masks were nearest neighbor-resampled to the VSASL scan resolution using AFNI's 3d_resample.

To reduce or minimize CSF contamination, the VSASL labels and controls were pair-wise subtracted in MATLAB R2021A (Natick, MA) to form a perfusion-weighted time series. Then the median absolute deviation (MAD) was computed voxel-wise across the time dimension, and the MAD values were thresholded at the 80th-percentile both within the whole-brain and also within each brain-slice to identify high-variance voxels contaminated by CSF. Voxels with MAD values above either threshold were excluded from the GM mask, and the resulting mask served as the final GM ROI for the subsequent analysis.

Data were denoised using an iterative denoising technique incorporating 2nd-order Fourier regressors (to preserve desired cardiac-driven fluctuations) and censoring volumes with outlier Γ values (e.g., due to finger motion). Supplementary details on denoising are available in Section S5.

### B.4 Data Analysis

For each VSASL scan, the denoised data were spatially averaged within the GM ROI to produce time series for control and label measurements, respectively. A cardiac phase ϕ was assigned to every data point via PPG-based retrospective gating. The control and label time series were separately fit to 2nd-order Fourier models based on prior work. The VSASL signal was then obtained by subtracting the control and label Fourier coefficients to yield:

$$S ( ϕ ) = {\hat{d}}_{0} + {\sum }_{k = 1}^{2} ( {\hat{d}}_{cos , k} cos ( ϕ ) + {\hat{d}}_{sin , k} sin ( ϕ ) ) , \qquad ( 8 )$$

where \hat{d}₀, \hat{d}_{cos,1}, \hat{d}_{sin,1}, \hat{d}_{cos,2} and \hat{d}_{sin,2} are the resultant Fourier coefficients of the signal S(ϕ). After obtaining S(ϕ), PI was quantified using Eq. 4. The stability of the PI measurements was then assessed using a residuals permutation approach. This procedure was repeated over 1000 iterations, and the resulting distribution of PI values was used to compute 95% confidence intervals. Additional details on the S(ϕ) computation and residuals permutation approach are provided in Section S2.

The measured PI values from the τ-stepping VSASL scans were fit to a version of Eq. 5 modified to account for heart rate variability:

$$PI ( τ ) = A \cdot κ ( τ ) \qquad ( 9 )$$

where κ(τ) is the average of

$$\vert sinc ( \frac{τ}{Γ} ) \vert$$

functions evaluated over all the Γᵢ's observed during the scanning session:

$$κ ( τ ) = \frac{1}{{N}_{T}} \cdot {{\sum }^{{N}_{Γ}}}_{i = 1} \vert sinc ( \frac{τ}{{Γ}_{i}} ) \vert \qquad ( 10 )$$

and N_{Γ} is the number of cardiac periods Γ observed. This effectively weights the model based on the distribution of Γᵢ's observed during the scanning session. The model fits were evaluated using R², as computed for an intercept-free model.

Using subjects with two runs of the τ=500 ms scan, the test-retest repeatability of the GM ROI PI measurement at τ=500 ms was assessed by using the Pearson correlation coefficient.

The association of PI with age across subjects was also assessed. Because PI depends on the ratio of τ/Γ, the comparison across subjects and conditions is facilitated by evaluating PI at a common reference ratio of τ/Γ=0.5 (or equivalently, σ=Γ/2), which was chosen to minimize the extrapolation from the τ=500 ms measurements (given observed cardiac periods around Γ=1000 ms). Based on Eq. 9:

$$PI ( τ = Γ / 2 ) = PI ( τ = 500 ms ) \cdot \frac{κ ( τ = Γ / 2 )}{κ ( τ = 500 ms )} \qquad ( 11 )$$

to yield a PI metric that was adjusted to individual cardiac periods and therefore comparable across subjects. For subjects with two τ=500 ms scans, the computation was performed twice and averaged the PI(τ=Γ/2) values. Then, the relationship between PI(τ=Γ/2) vs age was assessed using the Pearson correlation coefficient.

### B.5 Results

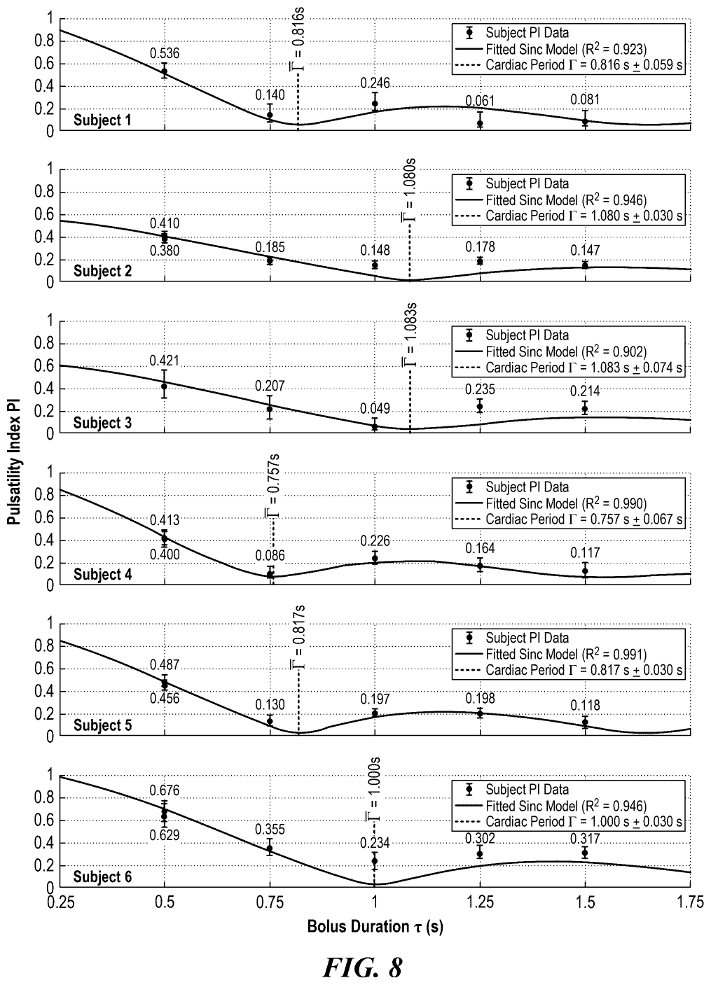

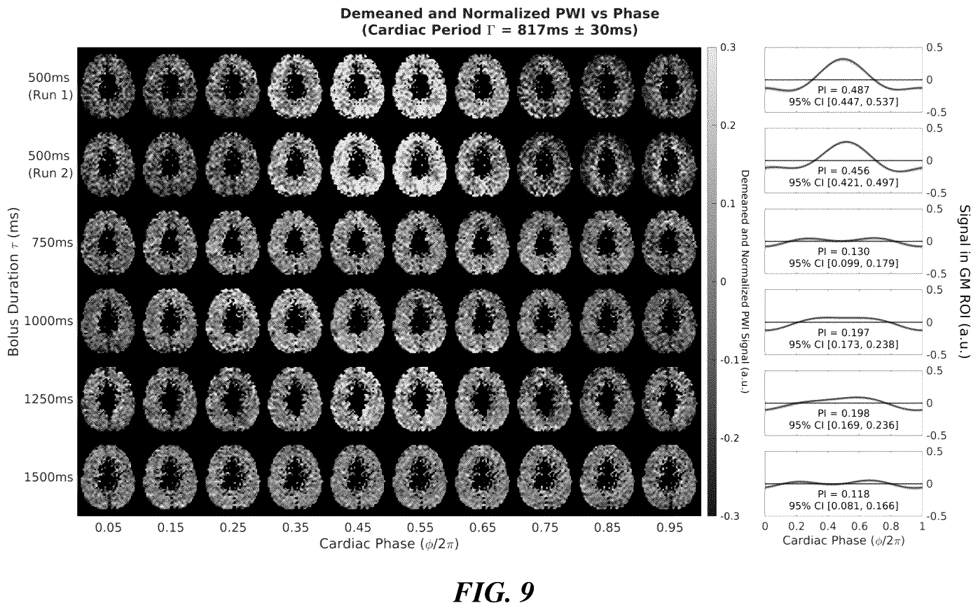

The fits of the subject data to the model in Eq. 9 are shown in FIG. 8, with R² values ranging from 0.902 to 0.991 and minima occurring near τ=Γ as predicted by Eq. 5. Notably, these subjects represent a diverse sample of cardiac periods, with values of Γ ranging from about 0.7 s to 1.1 s. FIG. 9 serves as a complementary figure demonstrating the dependence of PI on τ for a representative subject (Subject 5) with a cardiac period around Γ=817 ms. In both the images and the curves, the pulsatility observed in the τ=500 ms scans largely vanishes at τ=750 ms and τ=1500 ms, where the image intensities and signal curves flatten out across the cardiac cycle. Notably, the values of τ=750 ms and τ=1500 ms are close to Γ and 2Γ, respectively, where the

$$sinc ( \frac{τ}{Γ} )$$

term of Eq. 5 evaluates to 0.

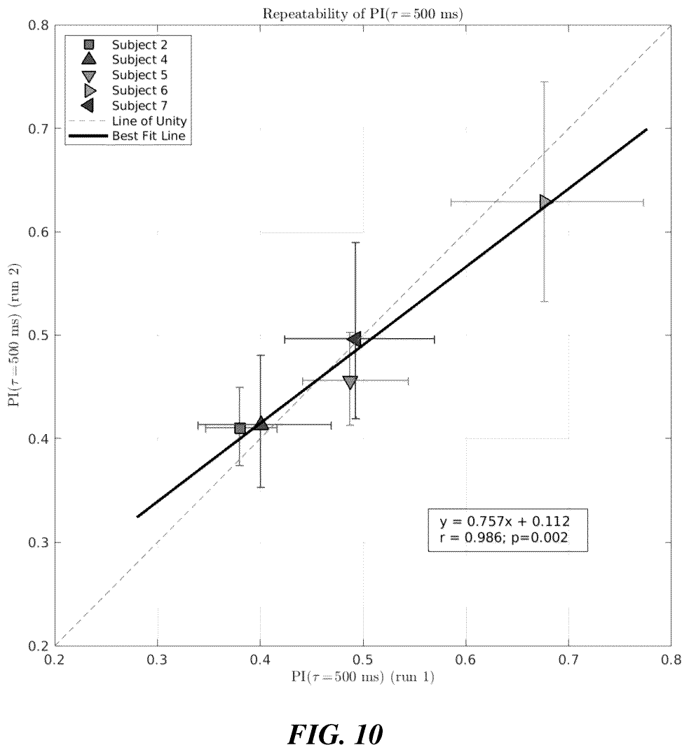

FIG. 10 shows the repeatability of PI at τ=500 ms across subjects. All data points lie close to the line of unity, with a high correlation coefficient (r=0.986, p=0.002). A representative visual example of this repeatability is demonstrated in FIG. 9 for subject 5, where the top two rows show the repeats at τ=500 ms. The appearances of the perfusion maps are nearly identical across the cardiac cycle. The morphologies of the GM ROI-averaged signal curves (FIG. 9, right) are also nearly identical, with (substantially) consistent shape and amplitude in both runs.

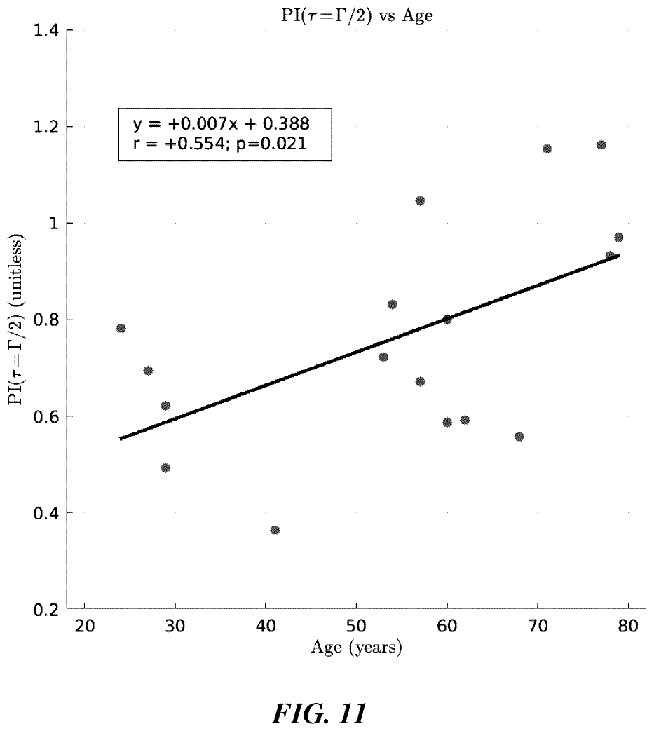

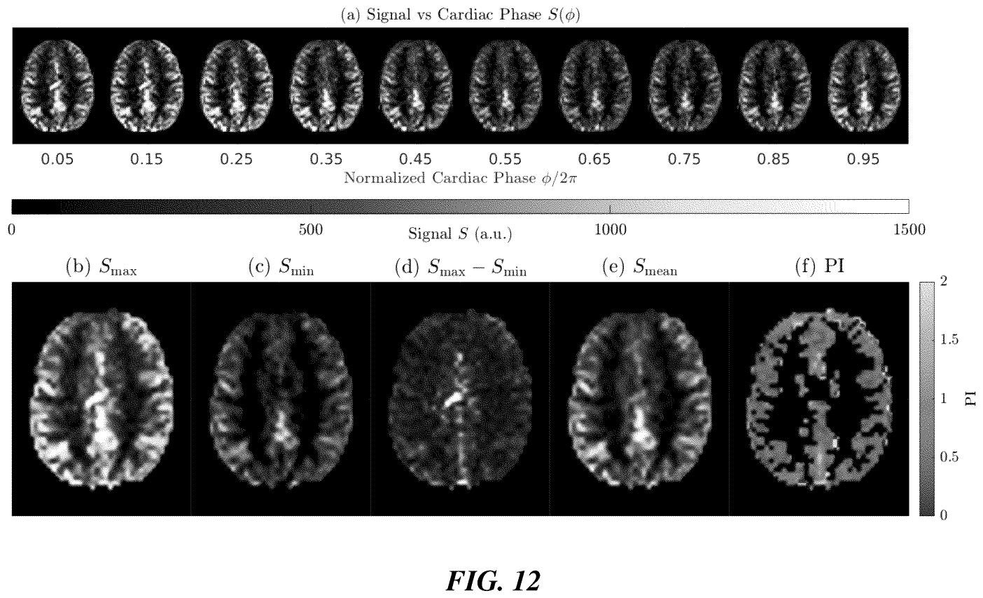

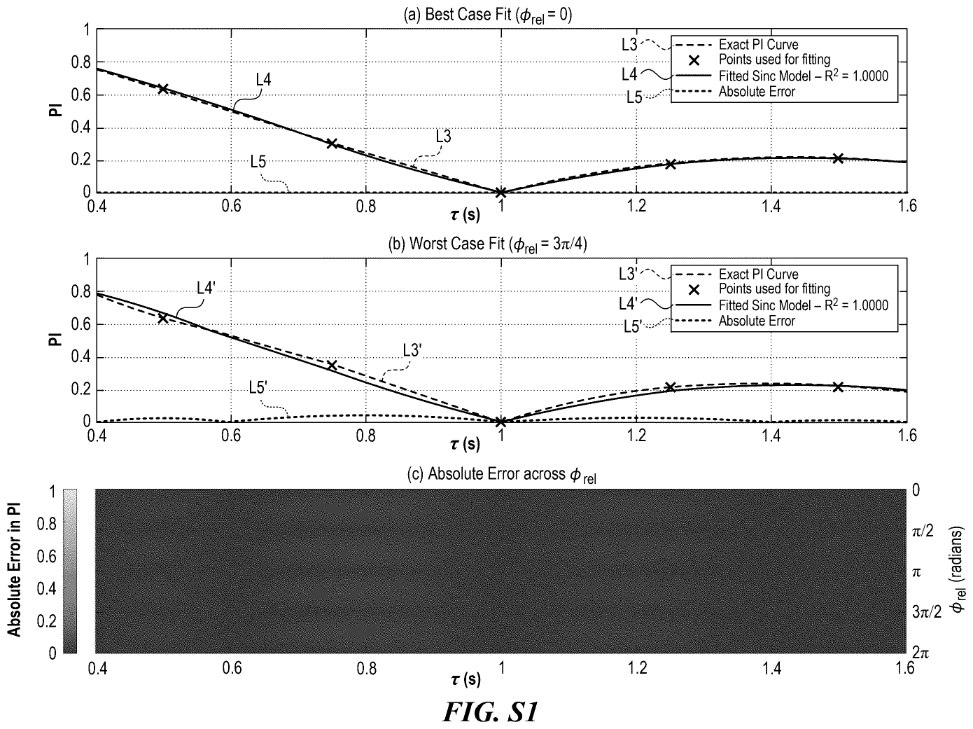

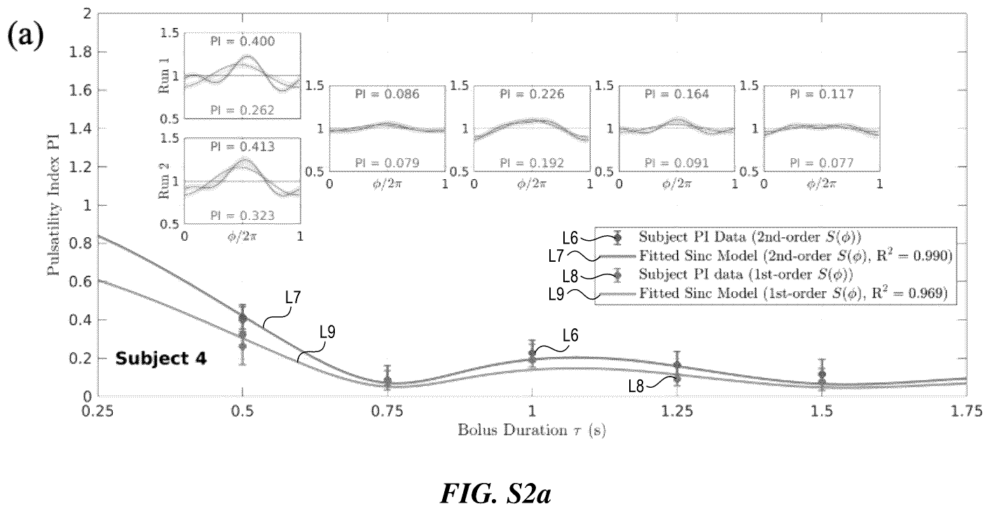

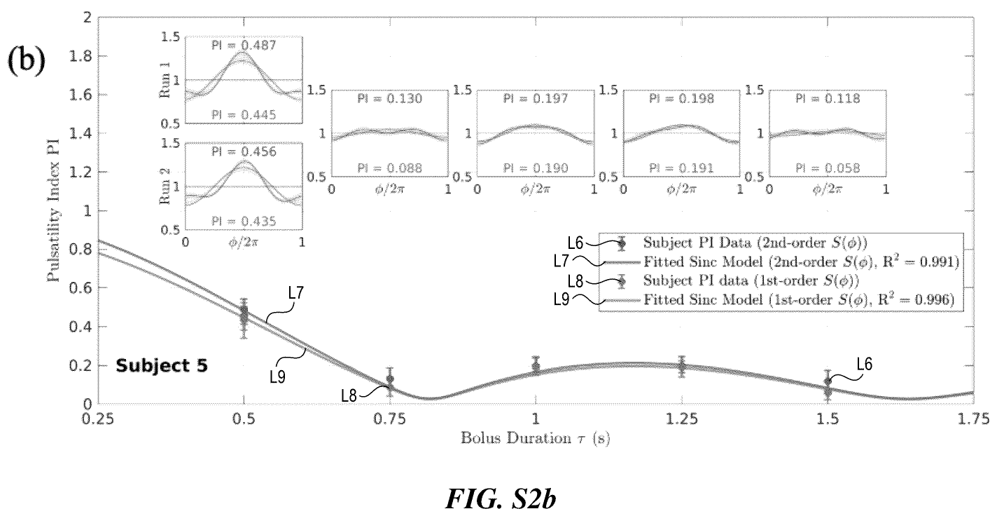

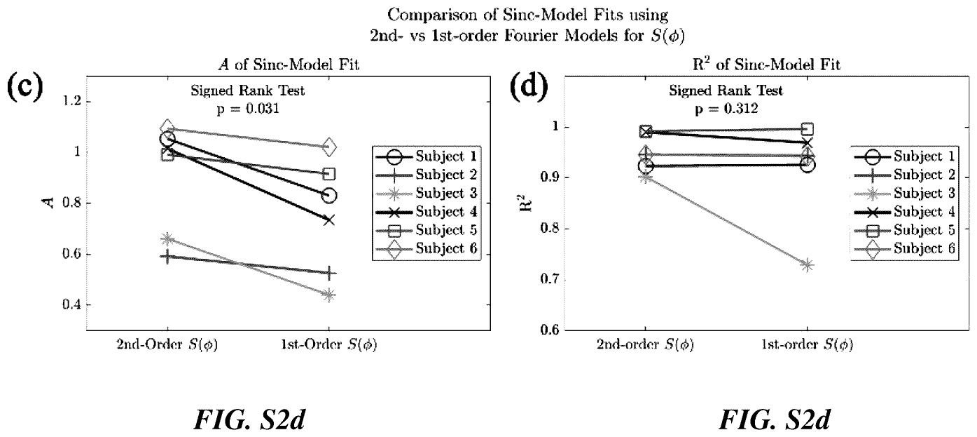

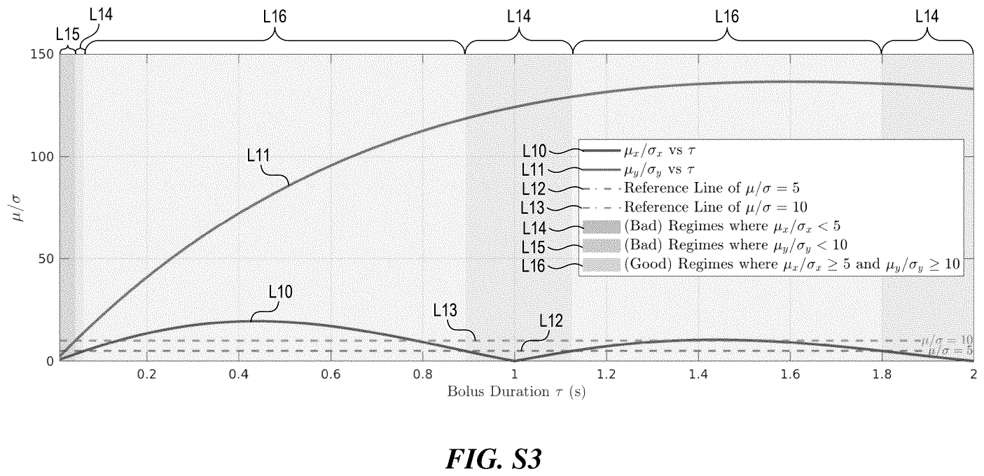

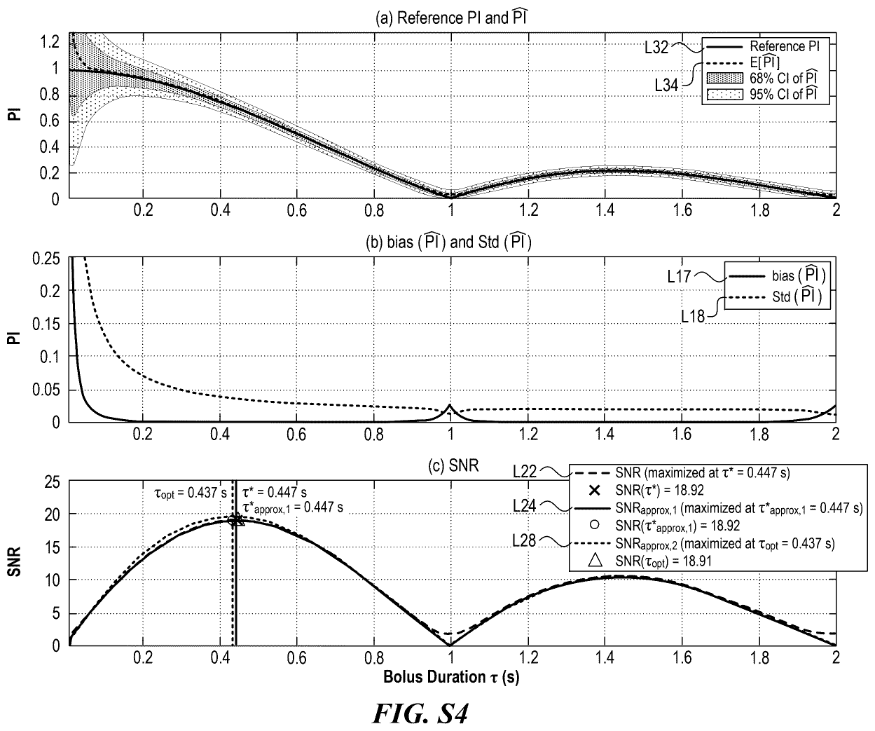

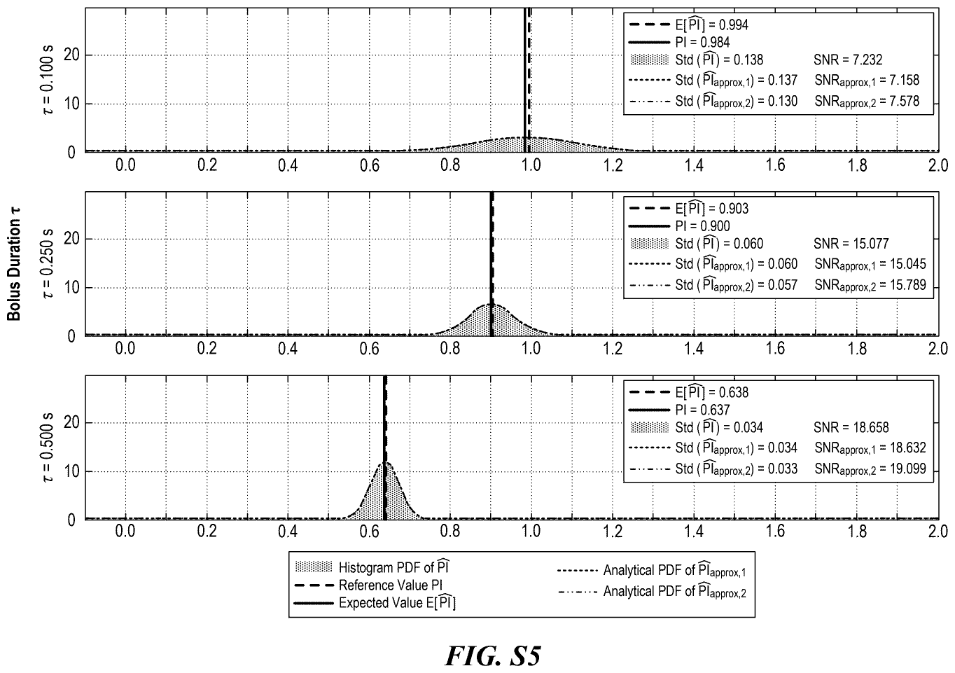

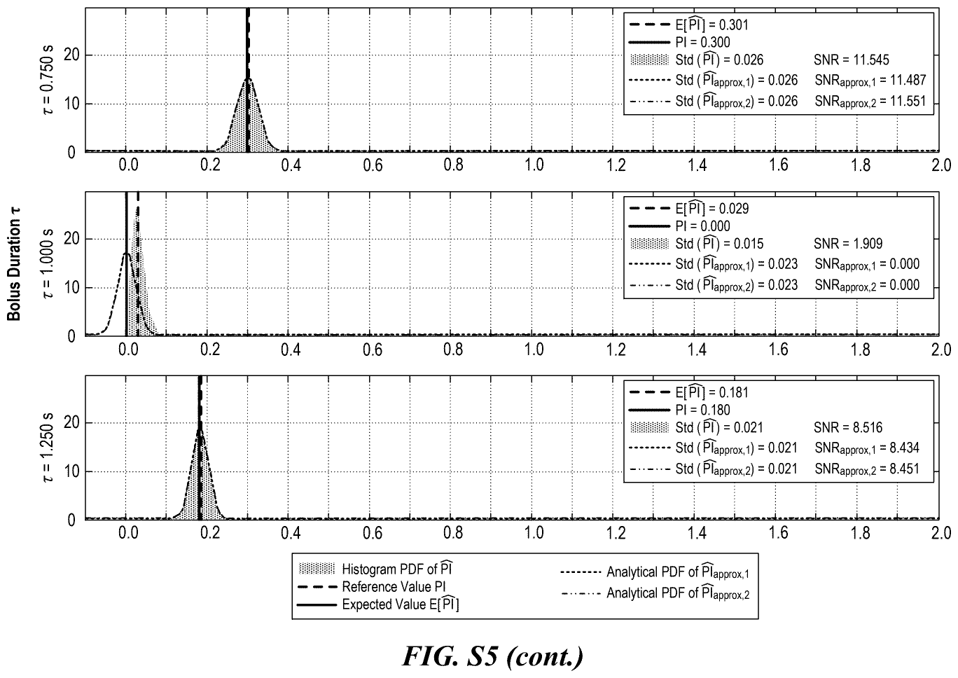

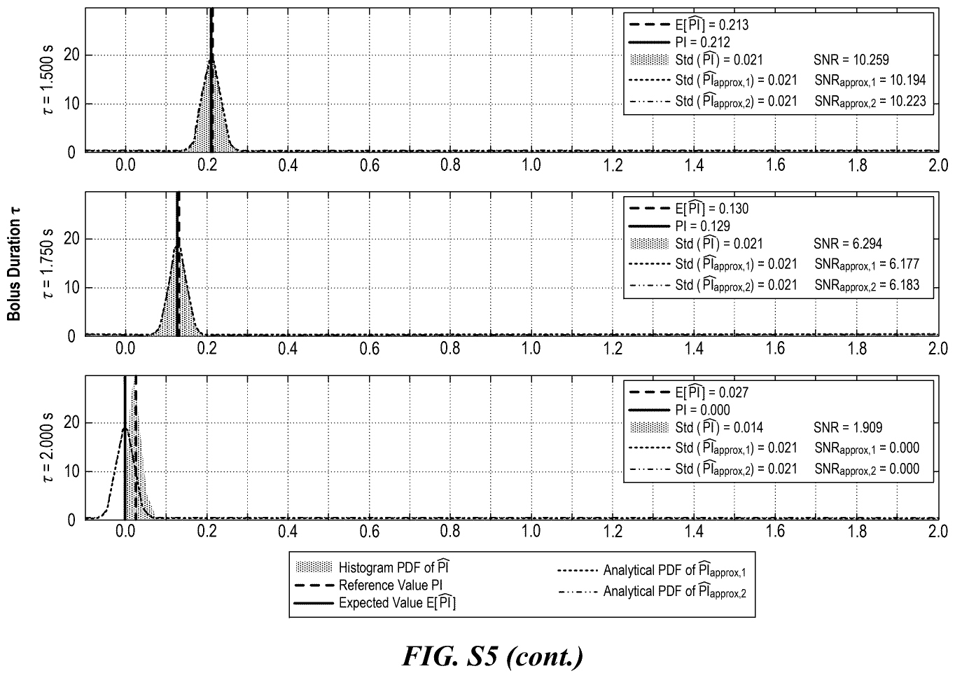

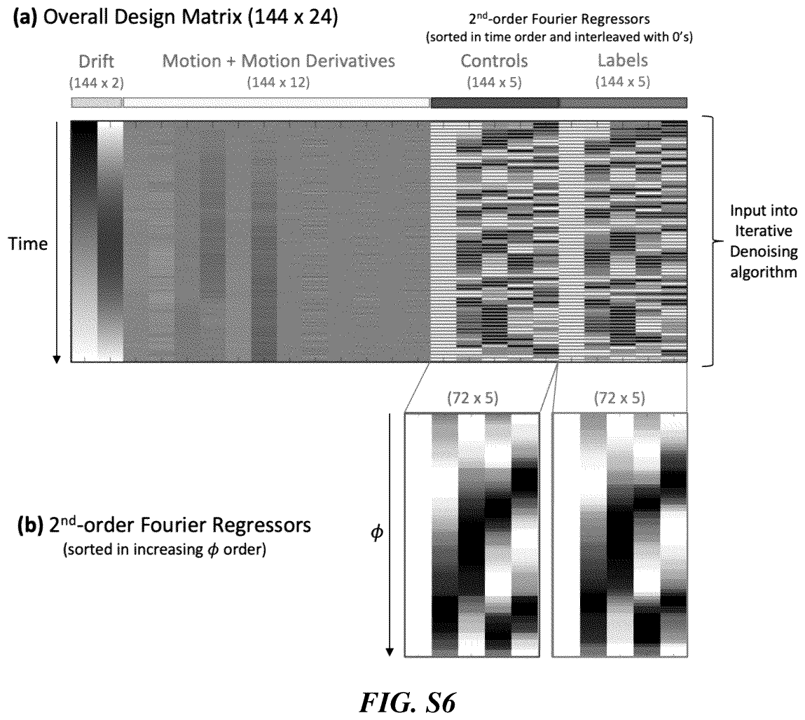

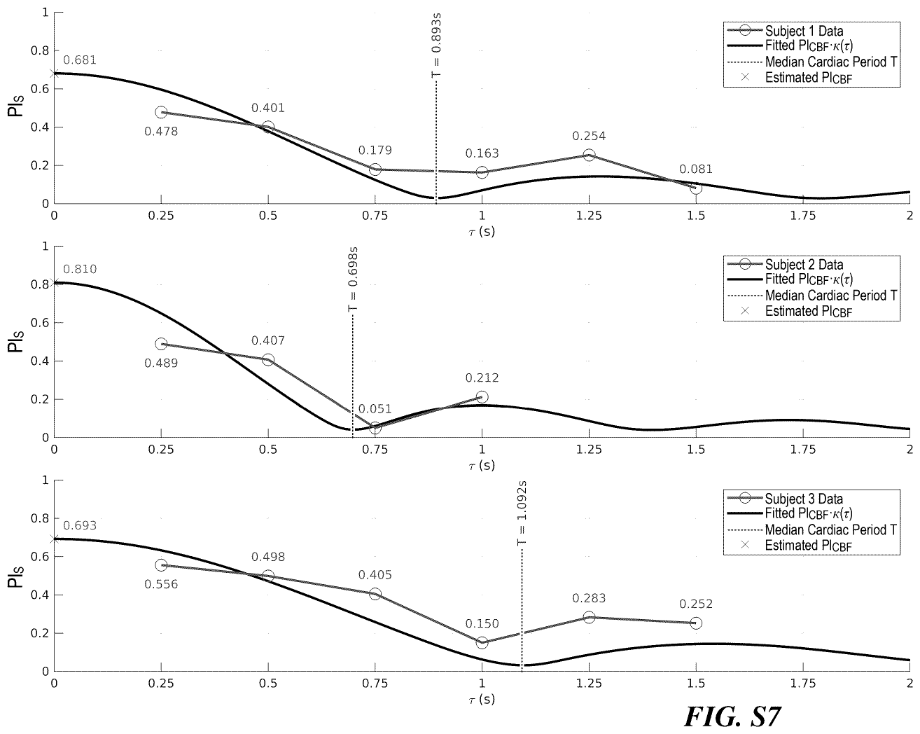

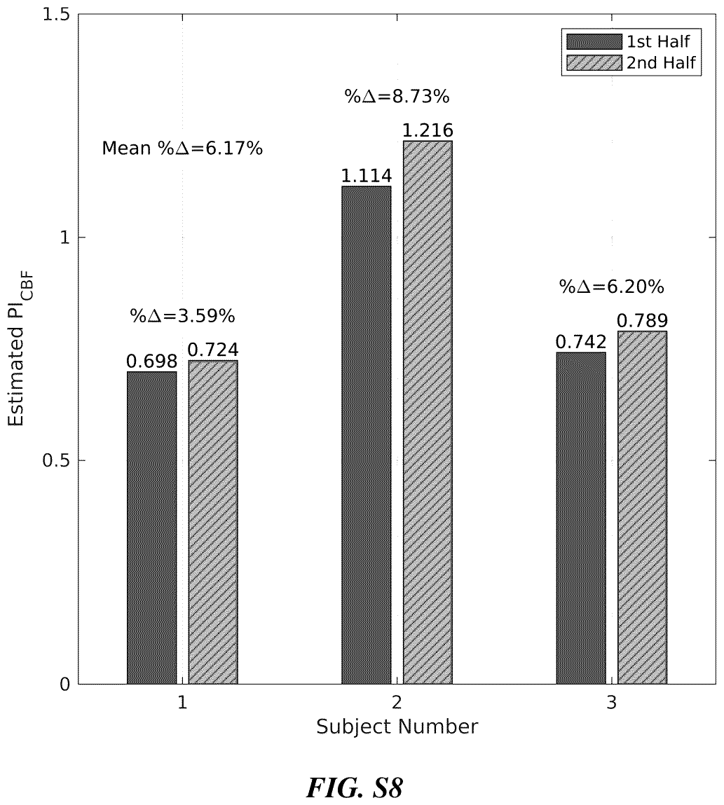

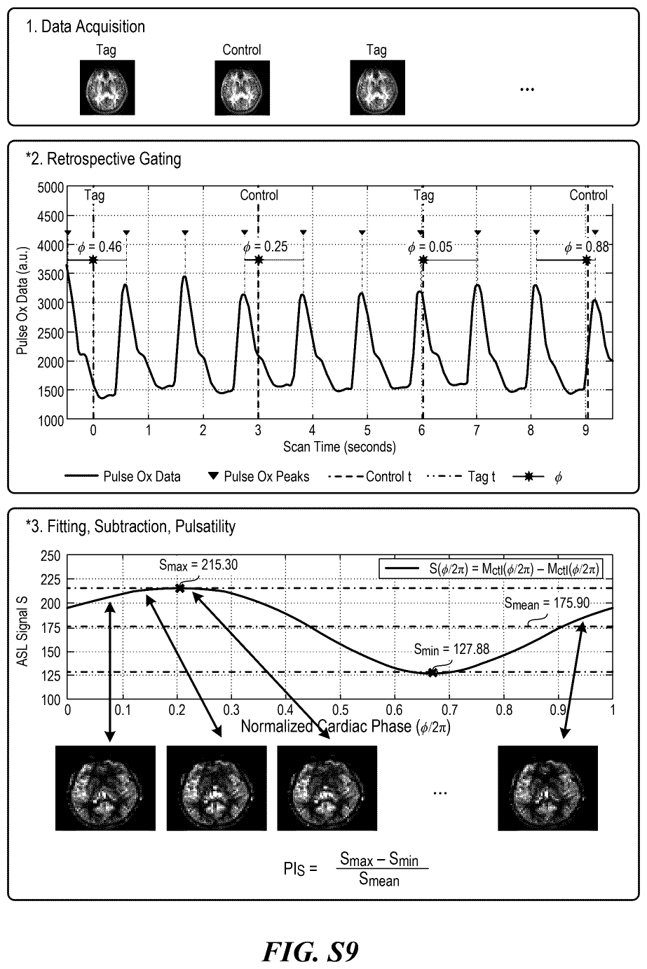

FIG. 11 shows a significant positive correlation between PI(τ=Γ/2) and age (r=0.554, p=0.021). FIG. 12 shows a representative voxel-wise PI map computed using data from subject 2. Following the same procedures followed for the GM ROI, the VSASL signal curve S(ϕ) was computed on a voxel-wise basis, followed by a voxel-wise PI computation via Eq. 4.

In summary, the theoretical model has showed excellent agreement to the empirical data in a gray matter region of interest (average R² value of 0.950±0.036 across six subjects (as illustrated in FIG. 8)). The results have showed excellent intrasession repeatability of the pulsatility measurement (r=0.986, p=0.002) and the potential to characterize associations with age (r=0.554, p=0.021). Finally, the results demonstrate the feasibility of whole-brain voxel-wise PI mapping, a contribution with potential for enabling regional pulsatility assessments.

### B.6 Discussion

The present document describes an approach for measuring cerebral microvascular pulsatility by leveraging the microvascular specificity of VSASL and/or retrospectively gating the VSASL signal. A key improvement of the current technology is the simple theoretical model for VSASL signal pulsatility PI given in Eq. 5, which provides an explicit form for PI as a function of bolus duration τ and cardiac period Γ. This sinc model is validated by its excellent fits to in vivo data from 6 subjects representing a broad range of cardiac periods. Furthermore, the model predicts a minimum in PI near τ=Γ, which is empirically observed in all subjects. This feature can be explained intuitively: A VSASL bolus of duration of τ=Γ integrates over one whole period of the periodic CBF signal, regardless of the position of the bolus in the cardiac cycle. This results in minimal fluctuation of the gated VSASL signal and thus a minimal value of PI.

Using this sinc model of Eq. 5, a theoretically optimal value of t may be obtained as shown in Eq. 7 to maximize the SNR of the pulsatility measurement. While the optimal value τₒₚₜ is slightly below Γ/2 based on theory, choosing τ=Γ/2 represents an SNR tradeoff of less than 3% for typical cardiac periods in the range of [0.6, 1.2] s. Thus, for researchers or clinicians seeking to use VSASL to measure microvascular pulsatility, choosing τ=Γ/2 can be a simple and effective rule of thumb to guide scan configurations.

The metric PI(τ=Γ/2) may be used for detecting physiological differences across subjects, as the positive association of PI(τ=Γ/2) with age (FIG. 11) agrees well with previous studies finding higher flow pulsatility (as measured by PC-MRI) in older vs younger subjects.

B.6.1 Comparison with Other Techniques

The strength of the present VSASL-based pulsatility approach is the ability to measure pulsatility in the microvasculature with vessel diameters around 50 μm. This is notably different from the majority of current pulsatility approaches, which focus on larger arteries feeding the brain. This focus on large vessels is a common feature across different modalities and techniques (e.g., 2D PC-MRI, 4D flow, doppler ultrasound, or ASL) and different measures of pulsatility (e.g., pulsatility of blood volume, vessel distension, flow velocity, or flow volume rate). For blood volume pulsatility, the one exception may be the dynamic ASL approach which can assess blood volume in vessels ranging from large arteries down to the capillaries, where no significant difference between systole and diastole was measured.

Another method that measures flow pulsatility in the microvasculature is PC-MRI at 7 T, which targets cerebral perforating arteries with diameters below 300 μm and flow velocities as low as 0.5-1.0 cm/s. This approach involves resolving and measuring the flow through individual vessels, averaging the flow curves across vessels, and computing pulsatility on the averaged flow signal. For an approximate comparison, the 7T PC-MRI microvascular pulsatility values in the basal ganglia (a deep gray matter region) were reported to be around 0.55−0.65 for young subjects (mean age around 25 years), and around 0.7−1.0 for older subjects (mean age around 60-65 years). Using an age cutoff of 45 years, PI(τ=Γ/2) values in cortical GM are 0.591±0.165 for younger subjects (n=5, age 30.0±6.48 years) and 0.835±0.217 for older subjects (n=12, age 64.7±9.56 years), which agree well with the 7T PC-MRI range of values. While the pulsatility values from 7T PC-MRI serve as a useful reference point, a direct comparison of their pulsatility values to those of the current study should be interpreted with caution due to (1) the different ROIs used between studies, and (2) the effect of bolus duration in the present VSASL approach. Specifically, the 7T PC-MRI studies have focused on the centrum semiovale (a white matter region) and basal ganglia, whereas the current VSASL study focuses on cortical GM. Future work assessing the same ROIs and exploring an appropriate adjustment for the τ-dependent sinc factor would be useful for a more direct comparison between the present VSASL method and the 7T PC-MRI approach.

The present VSASL approach builds upon the prior body of work examining cardiac-driven fluctuations and pulsatility of the ASL signal. While the majority of this literature focused on spatially-selective ASL techniques, others examined VSASL signal fluctuations primarily weighted by blood volume (due to the absence of a VCM), but did not explicitly compute a pulsatility index. Other ASL-based pulsatility measurements have used spatially-selective ASL approaches to assess cerebral blood volume (CBV) pulsatility and vascular compliance generally focusing on larger arteries, whereas the present VSASL approach focused on a flow pulsatility measurement in the microvasculature. For flow-weighted ASL signals, some studies recognized that selecting a bolus duration equal to the cardiac period (or a multiple) could mitigate cardiac-driven fluctuations, which are noted (both empirically and using theory) as well. However, the present document derives a model (Eq. 5) explicitly describing the dependence of cardiac fluctuations on τ and Γ, providing a useful theoretical extension to predict the magnitude of such signal fluctuations.

### B.6.2 Clinical Considerations

An accurate and reliable measurement of microvascular pulsatility can be valuable for clarifying the mechanisms connecting excessive pulsatility, microvascular damage, and cognitive decline in various diseases. Previous studies have associated decreased performance over a range of cognitive domains with pulsatility in large cerebral arteries, but these measurements remain distant from the microvasculature, where the damage linked to various cognitive disorders is likely occurring. In this regard, microvascular pulsatility measurements may be more suitable for assessing the relevant environment. Furthermore, since vascular risk factors such as microvascular pulsatility may reflect early and potentially modifiable variables in disease progression, the present technique may also help assess cognitive risk far before structural changes and clinical symptoms of cognitive impairment arise.

Because large arteries each supply pulsatile blood to extensive vascular territories that include many regions of the brain, the large-artery pulsatility indices are inherently limited in their spatial specificity when interpreting their impact on specific brain regions. In comparison, pulsatility measurements in the microvasculature, being much more distal along the arterial tree and embedded in the tissue they are supplying, can potentially be used to probe specific areas implicated in disease pathogenesis (e.g., the parietal lobe and hippocampus for Alzheimer's disease). The VSASL approach, with its whole-brain coverage and the ability to simultaneously generate microvascular signals across the entire brain, offers a unique potential to facilitate such regional assessments. FIG. 12 shows a voxel-wise cerebral pulsatility map determined by computing PI on a voxel-wise basis, illustrating its robustness and applicability to regional pulsatility assessments.

The technique has several practical features that may ease translation, including the ability to perform at 3 T, a simple prescription with whole-brain coverage, and a clinical scan time of around 6 minutes. These features may allow the VSASL pulsatility technique an attractive option for integration into standard clinical and research brain MRI protocols.

### B.6.3 Technical Considerations

In this study, the standard VSASL cutoff velocity of ν_{cut}=2 cm/s was used, which defines the VSASL bolus in the microvascular regime. However, the value of ν_{cut} can be adjusted to target other segments of the arterial network. Increasing ν_{cut} shifts the VSASL bolus-defining location more upstream into larger vessels, whereas decreasing ν_{cut} shifts the labeling region further distally toward the capillaries. Varying ν_{cut} may thus be useful for evaluating pulsatility along the arterial tree using a single approach and assessing metrics like dampening factor. Of note, decreasing ν_{cut} may need higher gradient strengths which can exacerbate known technical issues like eddy currents, diffusion effects, and CSF contamination. According to some embodiments, the technique disclosed herein may address the issue by employing a suitable VSASL pulse train (e.g., a suitable first VS pulse sequence and/or a suitable second VS pulse sequence).

Prior studies on VSASL have explored its potential to measure venous flow. The current technique may be applied to measure venous flow pulsatility as well.

The present approach may include features to reduce the CSF contamination of VSASL signals. This is an existing issue with VSASL in general as CSF is difficult to suppress through standard background suppression techniques. When using VSASL for quantifying mean cerebral blood flow, this manifests as an overestimation of perfusion in CSF-contaminated regions. CSF contamination may affect pulsatility measurements, as CSF can also pulsate with the cardiac cycle. The pre-processing attempts may include measures to mitigate CSF effects as much as possible by aggressively spatially excluding contaminated areas from the GM masks. Furthermore, these effects are challenging to remove through modeling due to the complex and heterogeneous nature of CSF flow. However, as VSASL sequences continue to improve, the approach as described herein can include other measures related to CSF suppression.

A standard BIR-8 train for VSASL labeling was used in this proof-of-principle study. The BIR-8 train is a velocity-selective saturation (VSS) technique noted for its robustness to B₀/B₁⁺ inhomogeneity and eddy currents, making it well suited for this initial study. Velocity-selective inversion (VSI) approaches may provide increased SNR and may also be used. The dynamic phase cycling method may be used to mitigate many of these or other artifacts.

### C. Conclusion

The present document describes a non-invasive approach of using VSASL to measure pulsatility in the microvasculature, including a theoretical framework relating the pulsatile VSASL signal to the pulsatility index. This technique may be used to probe questions on the mechanisms of cognitive disease (particularly in implicated regions), monitor disease progression, and evaluate patient responses to therapy.

### D. Tables and Figures

TABLE 1

| | | Number of | | Scan Duration |
|---|---|---|---|---|
| τ (ms) | BGS TI_{S} (ms) | L/C Pairs | TR (ms) | (mm:ss) |
| 500 | 137, 607 | 72 | 2450 | 6:17 |
| 750 | 137, 749 | 72 | 2700 | 6:39 |
| 1000 | 143, 929 | 70 | 2950 | 7:03 |
| 1250 | 138, 1111 | 64 | 3200 | 7:00 |
| 1500 | 137, 1283 | 60 | 3450 | 7:04 |

Table of VSASL scan parameters, indicating the bolus duration τ, background suppression inversion times (BGS TIs), number of label/control pairs, repetition time TR, and total scan duration. All scans used Tₛₐₜ=1500 ms and PLD=100 ms. Also, all VSASL scans contained one M₀ (proton density-weighted) repetition at the beginning of the sequence with TR=5000 ms. The BGS timings were configured via a MATLAB optimization routine to target complete suppression (0% signal) of GM and CSF tissue signals at 50 ms before the start of readout. This routine also considered T₂-decay during the label-control module (LCM) and vascular crushing module (VCM) assuming an effective echo time (eTE) of 22 ms, and T_{1,GM}=1300 ms, T_{2,GM}=100 ms, T_{1,CSF}=4000 ms, T_{2,CSF}=2000 ms.

TABLE 2

| Subject | Gender | Age (years) | Test-Retest | τ-Stepping |
|---|---|---|---|---|
| 1 | M | 24 | — | x |
| 2 | M | 41 | x | x |
| 3 | M | 29 | — | x |
| 4 | F | 27 | x | x |
| 5 | F | 29 | x | x |
| 6 | F | 57 | x | x |
| 7 | M | 60 | x | — |

Table of subject demographics and acquired data. Test-Retest refers to subjects who acquired two repeats of the τ=500 ms scan. τ-stepping refers to subjects who completed the τ-stepping protocol consisting of scans at τ=500/750/1000/1250/1500 ms. Only one τ=500 ms scan was acquired on Subject 1 due to time constraints. This was rectified with longer scheduled scan sessions for all subsequent subjects. Both τ=500 ms scans were acquired on Subject 3, but the first was excluded from analysis due to head motion. Subject 7 faced time constraints from late arrival, so only the two scans at τ=500 ms were collected.

FIG. 7: (a) Graphical representation of computed optimal tau τₒₚₜ following Eqs. 6 and 7, for an example cardiac period Γ=1 s. The dashed line L1 indicates the VSASL signal SNR model τ·exp(−t/T1)). The dashed line L2 indicates the PI sinc model of Eq. 5. The solid line indicates the combined PI SNR model (Eq. 6) balancing between VSASL SNR and PI. This balance is optimized at τₒₚₜ (computed via Eq. 7), which is indicated by the purple open circle. (b) A plot of τₒₚₜ vs Γ per Eq. 7. Several points are highlighted to indicate τₒₚₜ for values of Γ in a reasonable physiological range. By evaluating the PI SNR model (Eq. 6) at τₒₚₜ and Γ/2, the discrepancy between SNR(τₒₚₜ) and SNR(Γ/2) is shown to be fairly small (<3% for Γ in [0.6, 1.2] s), suggesting Γ/2 is a good rule of thumb for selecting Γ for a pulsatility measurement.

FIG. 8: Fits of the sinc model (Eq. 9) to the PI values measured from the τ-stepping protocol on each subject. The blue data points indicate the PI measured at each τ scan, with the black curve representing the fitted sinc model of Eq. 9. The model fits the data quite well across a range of heart rates represented by these subjects. PI may reach a minimum around τ=Γ as predicted by theory in Eq. 5.

FIG. 9: The images show the demeaned and normalized perfusion-weighted image (PWI) signal vs ϕ/2π for a select slice of Subject 5. The left-hand montage shows demeaned and normalized perfusion-weighted images (PWI), with cardiac cycle along the x-axis and τ values for each scan indicated along the y-axis. To produce the maps on the left-hand side, the data are fitted voxel-wise to obtain S(ϕ) curves for every voxel, then voxel-wise demeaned, and finally normalized by the scalar GM ROI mean value. The voxel intensities thus represent the relative fluctuation amplitude around each voxel's mean compared to the baseline GM value. The voxels in the middle of the slices were identified as high-MAD voxels (cerebrospinal fluid (CSF) contaminated) in Section B.3 and were masked out. The accompanying line plots on the right-hand side show the GM ROI-averaged signal S(ϕ). The red shaded regions represents the 95% confidence interval at each value of ϕ/2π, derived from the set of S(ϕ) curves resulting from the residuals permutation approach. The text indicates the PI value, along with its 95% confidence interval. Note that for better visual comparison, the curves (and images) were phase shifted so that the maximum amplitude roughly aligns across the scans (rows) in this figure.

FIG. 10: Repeatability of PI(τ=500 ms) across subjects. The x-axis denotes the PI of run 1, and the y-axis denotes the PI of run 2. The data points are the measured values from the 5 subjects with repeats of the τ=500 ms scan. The error bars indicate the 95% confidence intervals of the values. The values follow the line of unity closely, indicating excellent test-repeat repeatability. This is further supported by the high correlation coefficient r=0.986 (p=0.002).

FIG. 11: Plot of PI(τ=Γ/2) vs age, showing a positive correlation between PI(τ=/2) and age (r=+0.554, p=0.021).

FIG. 12: Demonstration of a voxel-wise PI map, computed on Subject 2, τ=500 ms, run 1. (a) Perfusion signal as a function of cardiac phase S(ϕ), computed as in Section B.4 on a voxel-wise basis. The subsequent panels show the individual components of the Eq. 4 formula, where (b) shows Sₘₐₓ, (c) shows Sₘᵢₙ, (d) shows Sₘₐₓ−Sₘᵢₙ (the numerator of the PI formula), (e) shows Sₘₑₐₙ (the denominator of the PI formula), and (f) shows the final PI map. Note that the image in (f) has been masked to focus on gray matter voxels.

### Supporting Information:

### S1 Evaluating Sinc Model Approximation

In this section, the error associated with the sinc model of pulsatility (Eq. 5) may be evaluated, whose derivation involves neglecting the 2nd-order Fourier terms. To evaluate the error, an exact expression for PI(τ) of a 2nd-order Fourier signal is derived. Then the exact PI(τ) curves (the reference) are computed over a range of τ values, and Eq. 5 (the model) is fit to the reference to evaluate the model error.

### S1.1 Theory

To provide an exact expression of PI, the 2nd-order Fourier model for S(t) is given in Eq. 3, with the summation expanded out for clarity in this section (the T₁ exponential decay factor is excluded for simplicity as it cancels out in the final PI calculation).

$$\begin{matrix}S ( t ) ∝ {b}_{0} τ + {b}_{cos , 1} τ \cdot sinc ( \frac{τ}{Γ} ) \cdot cos ( \frac{2 π t}{Γ} ) + {b}_{sin , 1} τ \cdot sinc ( \frac{τ}{Γ} ) \cdot sin ( \frac{2 π t}{Γ} ) \\ + {b}_{cos , 2} τ \cdot sinc ( \frac{2 τ}{Γ} ) \cdot cos ( \frac{4 π t}{Γ} ) + {b}_{sin , 2} τ \cdot sinc ( \frac{2 τ}{Γ} ) \cdot sin ( \frac{4 π t}{Γ} )\end{matrix} \qquad ( S \text{.1} )$$

By combining the cos(·) and sin(·) terms of the same order, the equation is rewritten as:

$$\begin{matrix}S ( t ) ∝ {b}_{0} τ + τ \cdot sinc ( \frac{τ}{Γ} ) [ {W}_{1} \cdot cos ( \frac{2 π t}{Γ} - arctan ( \frac{{b}_{sin , 1}}{{b}_{cos , 1}} ) ) ] \\ + τ \cdot sinc ( \frac{2 τ}{Γ} ) [ {W}_{2} \cdot cos ( \frac{4 π t}{Γ} - arctan ( \frac{{b}_{sin , 2}}{{b}_{cos , 2}} ) ) ]\end{matrix} \qquad ( S \text{.2} )$$

$$where$$

$$\begin{matrix}{W}_{1} = \sqrt{{b}_{cos , 1}^{2} + {b}_{sin , 1}^{2}} \\ {W}_{2} = \sqrt{{b}_{cos , 2}^{2} + {b}_{sin , 2}^{2}}\end{matrix} \qquad ( S \text{.3} )$$

represent the magnitudes of the 1st and 2nd-order Fourier components, respectively. To compute PI, Eq. 4 (reproduced below) may be used:

$$PI = \frac{{S}_{max} - {S}_{min}}{{S}_{mean}} \qquad ( S \text{.4} )$$

by evaluating the individual terms of the formula. The mean term Sₘₑₐₙ is simply the constant term:

$${S}_{mean} = {b}_{0} τ . \qquad ( S \text{.5} )$$

The max term Sₘₐₓ is computed by taking the maximum of S(t):

$$\begin{matrix}{S}_{max} = {b}_{0} τ + τ \cdot max [ {W}_{1} \cdot sinc ( \frac{τ}{Γ} ) cos ( \frac{2 π t}{Γ} - arctan ( \frac{{b}_{sin , 1}}{{b}_{cos , 1}} ) ) … \\ + {W}_{2} \cdot sinc ( \frac{2 τ}{Γ} ) cos ( \frac{4 π t}{Γ} - arctan ( \frac{{b}_{sin , 2}}{{b}_{cos , 2}} ) ) ] .\end{matrix} \qquad ( S \text{.6} )$$

Because the max[·] operation is insensitive to phase shifts, Sₘₐₓ can be further rewritten as:

$${S}_{max} = {b}_{0} τ + τ \cdot max [ {W}_{1} \cdot sinc ( \frac{τ}{Γ} ) cos ( \frac{2 π t}{Γ} - {ϕ}_{rel} ) + {W}_{2} \cdot sinc ( \frac{2 τ}{Γ} ) cos ( \frac{4 π t}{Γ} ) ] \qquad ( S \text{.7} )$$

where ϕᵣₑₗ is defined as:

$${ϕ}_{rel} = arctan ( \frac{{b}_{sin , 1}}{{b}_{cos , 1}} ) - \frac{1}{2} \cdot arctan ( \frac{{b}_{sin , 2}}{{b}_{cos , 2}} )$$

and represents the relative phase shift between 1st- and 2nd-order sinusoids. Following the same logic, the min term Sₘᵢₙ can be represented in a similar way:

$${S}_{min} = {b}_{0} τ + τ \cdot min [ {W}_{1} \cdot sinc ( \frac{τ}{Γ} ) cos ( \frac{2 π t}{Γ} - {ϕ}_{rel} ) + {W}_{2} \cdot sinc ( \frac{2 τ}{Γ} ) cos ( \frac{4 π t}{Γ} ) ] . \qquad ( S \text{.9} )$$

By substituting Sₘₑₐₙ, Sₘₐₓ and Sₘᵢₙ into Eq. 4, the following exact expression for PI(τ) may be obtained:

$$\begin{matrix}PI = ( max [ {W}_{1} \cdot sinc ( \frac{τ}{Γ} ) cos ( \frac{2 π t}{Γ} - {ϕ}_{rel} ) + {W}_{2} ( \frac{2 τ}{Γ} ) cos ( \frac{4 π t}{Γ} ) ] \\ - min [ {W}_{1} \cdot sinc ( \frac{π}{Γ} ) cos ( \frac{2 π t}{Γ} - {ϕ}_{rel} ) + {W}_{2} \cdot sinc ( \frac{2 τ}{Γ} ) cos ( \frac{4 π t}{Γ} ) ] ) / {b}_{0}\end{matrix} \qquad ( S \text{.10} )$$

This is a formula for PI that depends on the scaling terms b₀, W₁, W₂ and a relative phase shift between 1st- and 2nd-order cosines given by ϕᵣₑₗ. Note that if the 2nd-order component is neglected by setting W₂=0, then the formula simplifies down to the sinc model stated in Eq. 5 of the main text and reproduced below for convenience:

$$PI ( τ ) = A \cdot \vert sinc ( \frac{τ}{Γ} ) \vert \qquad ( S \text{.11} )$$

where

$$A = 2 {W}_{1} / {b}_{0} = 2 \sqrt{{b}_{cos , 1}^{2} + {b}_{sin , 1}^{2}} / {b}_{0}$$

is the lumped fitting parameter described in the main text.

### S1.2 Methods

To evaluate the errors associated with the sinc model approximation, we: (1) computed exact PI(τ) curves (Eq. S.10), (2) fit the sinc model (Eq. 5) to those exact curves, and (3) assessed the error of those fits.

To produce the exact PI(τ) curves, the constants (b₀, W₁, W₂) were set at “reasonable” values based on empirical measurements of the present work and on prior literature. First, b₀=1 and W₁=0.5 were chosen to ensure the PI(τ) curves have values comparable to in vivo data measurements at τ=500/750/1000/1250/1500 ms. While the exact b₀ and W₁ values are arbitrary, their ratio controls the vertical scaling factor

$$A = 2 {W}_{1} / {b}_{0} = 2 \sqrt{{b}_{cos , 1}^{2} + {b}_{sin , 1}^{2}} / {b}_{0}$$

in the Eq. 5 sinc model, which roughly scales the exact PI(τ) curves as well. To choose W₂, prior estimates of blood flow power spectra in the internal carotid artery (ICA) were used to assume the power ratio of the 1st-vs 2nd-order components

$$( {W}_{1}^{2} / {W}_{2}^{2} )$$

as 5:1, which sets W₂=W₁/\sqrt{5}=0.5/\sqrt{5}=0.2236. (Note that the power ratio in the microvasculature may even be higher than 5:1; arterial compliance generally behaves like a low-pass filter on flow waveforms, which would further attenuate the 2nd-order power as blood travels from the ICA to the microvasculature. A higher ratio would mean an even smaller relatively W₂ value, or a weaker 2nd-order component.) To summarize, the constants are set at b₀=1, W₁=0.5 and W₂=0.2236.

A cardiac period of Γ=1 s was set based on the range observed in subjects. Bolus duration τ was varied over τ∈[0.400, 1.600] s in steps of 0.001 s. The remaining unconstrained parameter of ϕᵣₑₗ (the relative phase shift between 1st- and 2nd-order components) was varied between 0 to 2π in steps of 2π·0.001.

For each combination of ϕᵣₑₗ and τ, the exact value of PI(τ) (Eq. S.10) was computed by numerically evaluating its terms over t∈[0, Γ] in steps of 0.0001 s. This resulted in curves of PI(τ) for every value of ϕᵣₑₗ. Finally, for each value of ϕᵣₑₗ, the sinc model (Eq. 5) was fit to the exact PI values at τ=500/750/1000/1250/1500 ms, which correspond to the τ values used in the τ-stepping experiment.

Since the derivation of the sinc model (Eq. 5) involves neglecting 2nd-order terms, a natural question is whether a 1st-order Fourier model of the signal S may be used to start with, which may obviate the need for any approximation step in the theory. As a complementary empirical analysis, the τ-stepping data (FIG. 8 of the main text) are re-analyzed by using a 1st-order Fourier model to describe S(ϕ) and compared the results to those of the 2nd-order approach. The same procedures outlined in Section B.4 were followed, with the exception of using a 1st-order Fourier model to fit the controls and labels data, which resulted in a 1st-order model of S(ϕ). The S(ϕ) curves for the 2nd-vs 1st-order approaches were qualitatively compared. After fitting the τ-stepping data to the sinc model (Eq. 9), the resulting values of A (i.e., the scaling factor of the sinc model) and R² of the fits were also compared between the 2nd-vs 1st-order approaches using a paired Wilcoxon signed rank test.

### S1.3 Results

The results of the fits are shown in FIG. S1, with diagram (a) of FIG. S1 showing the best case fit based on R² (over values of ϕᵣₑₗ), diagram (b) of FIG. S1 showing the worst case fit, and diagram (c) of FIG. S1 the error across the entire space of ϕᵣₑₗ. There is very little error across the entire space of τ and ϕᵣₑₗ, supporting the validity of the sinc model (Eq. 5) for describing pulsatility as a function of τ.

FIG. S2 shows the results of the empirical τ-stepping analysis comparison. As shown in the insets of FIG. S2a and FIG. S2b, the main qualitative difference between the 2nd-vs 1st-order approaches is that the 1st-order model of S(ϕ) has difficulty describing relatively “sharper” peaks (which are captured by the 2nd-order approach), an effect most clearly shown for the τ=500 ms scans. As a result, the 1st-order approach tends to underestimate PI for each t scan, which can be seen in the individual subject examples (the orange data points in L8 and L9 being consistently lower than the blue ones in L6 and L7). This effect is also summarized in FIG. S2c, where the estimated scaling parameter A of the sinc model (Eq. 9) is lower with the 1st-order approach for all six subjects. However, the ability of the sinc model to describe the PI(τ) data seems to remain excellent regardless of the 2nd-vs 1st-order approach taken. As shown in FIG. S2d, the R2 values of the fits are all >0.9 (with one outlier exception in Subject 3 for the 1st-order approach), and the Wilcoxon signed rank test shows no statistically significant difference between the R² values (p=0.312). Overall, while using the 1st-order approach of S(ϕ) seems to underestimate the PI values, the sinc model still appears to describe the PI(τ) shape well with either approach based on these initial comparisons.

### S2 Computing VSASL Signal and Pulsatility

This section provides supporting details for Section B.4 of the main text, as described in the computation of the VSASL signal as a function of cardiac phase denoted as S(ϕ) and the subsequent PI and confidence interval computations.

The computation of S(ϕ) involves (1) retrospective gating to map control and label data (acquired in time) to cardiac phase space, (2) fitting 2nd-order Fourier models to control and label data points, and (3) subtracting the fits to obtain S(ϕ). Starting with (N×1) control and label measurement vectors:

$$\begin{matrix}{Y}_{C} = {[ {y}_{C , 1} {y}_{C , 2} … {y}_{C , N} ]}^{T} ( N \times 1 ) \\ {Y}_{L} = {[ {y}_{L , 1} {y}_{L , 2} … {y}_{L , N} ]}^{T} ( N \times 1 )\end{matrix} \qquad ( S \text{.12} )$$

where y_{C,i} and y_{L,i} represent the ith control and label measurements, respectively, and N is the number of control/label pairs acquired from the scan. These measurement vectors can represent an ROI-averaged signal (as in the GM ROI-averaged signal used in Section B.3) or the values from single voxels (as was used to produce the voxelwise PI map in FIG. 12).

Next, a cardiac phase ϕ was assigned to every label and control data point by retrospectively gating on the photoplethysmography (PPG) waveform trace. First the peaks of the PPG waveforms were detected. Then, a cardiac phase ϕ_{C,i} or ϕ_{L,i} was assigned to every control and label volume, respectively, using:

$$ϕ = 2 π \cdot ( \frac{t - {t}_{1}}{{t}_{2} - {t}_{1}} ) \qquad ( S \text{.13} )$$

where t is evaluated at t_{C,i} or t_{L,i} (defined as the center of the labeling/control period with width t), t₁ is the preceding cardiac peak, and t₂ is the subsequent cardiac peak. For a given data point, time t_{C,i} or t_{L,i} was computed by subtracting PLD+Γ/2 from the start time of the readout. Vectors of these ϕ were constructed for the controls and labels:

$$\begin{matrix}{ϕ}_{C} = {[ {ϕ}_{C , 1} {ϕ}_{C , 2} … {ϕ}_{C , N} ]}^{T} ( N \times 1 ) \\ {ϕ}_{L} = {[ {ϕ}_{L , 1} {ϕ}_{L , 2} … {ϕ}_{L , N} ]}^{T} ( N \times 1 )\end{matrix} \qquad ( S \text{.14} )$$

where ϕ_{C,i} and ϕ_{L,i} represent the cardiac phase of the ith control and label measurements, respectively. Then 2nd-order Fourier design matrices were constructed as:

$$\begin{matrix}{X}_{C} = [ \begin{matrix}1 & cos ( {ϕ}_{C , 1} ) & sin ( {ϕ}_{C , 1} ) & cos ( 2 {ϕ}_{C , 1} ) & sin ( 2 {ϕ}_{C , 1} ) \\ 1 & cos ( {ϕ}_{C , 2} ) & sin ( {ϕ}_{C , 2} ) & cos ( 2 {ϕ}_{C , 2} ) & sin ( 2 {ϕ}_{C , 2} ) \\ ⋮ & ⋮ & ⋮ & ⋮ & ⋮ \\ 1 & cos ( {ϕ}_{C , N} ) & sin ( {ϕ}_{C , N} ) & cos ( 2 {ϕ}_{C , N} ) & sin ( 2 {ϕ}_{C , N} )\end{matrix} ] ( N \times 5 ) \\ {X}_{L} = [ \begin{matrix}1 & cos ( {ϕ}_{L , 1} ) & sin ( {ϕ}_{L , 1} ) & cos ( 2 {ϕ}_{L , 1} ) & sin ( 2 {ϕ}_{L , 1} ) \\ 1 & cos ( {ϕ}_{L , 2} ) & sin ( {ϕ}_{L , 2} ) & cos ( 2 {ϕ}_{L , 2} ) & sin ( 2 {ϕ}_{L , 2} ) \\ ⋮ & ⋮ & ⋮ & ⋮ & ⋮ \\ 1 & cos ( {ϕ}_{L , N} ) & sin ( {ϕ}_{L , N} ) & cos ( 2 {ϕ}_{L , N} ) & sin ( 2 {ϕ}_{L , N} )\end{matrix} ] ( N \times 5 )\end{matrix} \qquad ( S \text{.15} )$$

which can also be more compactly written using the cardiac phase column vectors:

$$\begin{matrix}{X}_{C} = [ \begin{matrix}\vert & \vert & \vert & \vert & \vert \\ 1 & cos ( {ϕ}_{C} ) & sin ( {ϕ}_{C} ) & cos ( 2 {ϕ}_{C} ) & sin ( 2 {ϕ}_{C} ) \\ \vert & \vert & \vert & \vert & \vert\end{matrix} ] ( N \times 5 ) \\ {X}_{L} = [ \begin{matrix}\vert & \vert & \vert & \vert & \vert \\ 1 & cos ( {ϕ}_{L} ) & sin ( {ϕ}_{L} ) & cos ( 2 {ϕ}_{L} ) & sin ( 2 {ϕ}_{L} ) \\ \vert & \vert & \vert & \vert & \vert\end{matrix} ] ( N \times 5 )\end{matrix} \qquad ( S \text{.16} )$$

To fit Y_{C} and Y_{L} to separate 2nd-order Fourier models, the data are expressed as two over-determined systems of linear equations:

$$\begin{matrix}{Y}_{C} = {X}_{{C}^{C}} \\ {Y}_{L} = {X}_{L} 1\end{matrix} \qquad ( S \text{.17} )$$

where c and l are the 2nd-order Fourier coefficients for controls and labels, respectively. They are estimated via least squares:

$$\begin{matrix}\hat{c} = {( {X}_{C}^{T} {X}_{C} )}^{- 1} {X}_{C}^{T} {Y}_{C} \\ \hat{l} = {( {X}_{L}^{T} {X}_{L} )}^{- 1} {X}_{L}^{T} {Y}_{L}\end{matrix} \qquad ( S \text{.18} )$$

with ĉ=[ĉ₀ ĉ_{cos,1} ĉ_{sin,1} ĉ_{cos,2} ĉ_{sin,2}]^{T} and Î=[\hat{l}_{cos,1} \hat{l}_{sin,1} \hat{l}_{cos,2} \hat{l}_{sin,2}]^{T} denoting the corresponding estimates. The difference (signal S) coefficients are then computed by control-minus-label subtraction:

$$\hat{d} = \hat{c} - \hat{l} \qquad ( S \text{.19} )$$

where \hat{d}=[\hat{d}₀ \hat{d}_{cos,1} \hat{d}_{sin,1} \hat{d}_{cos,2} \hat{d}_{sin,2}]^{T}. The signal curve S(ϕ) can then be obtained by inserting the coefficients into a 2nd-order Fourier model (Eq. 8 of the main text, reproduced below):

$$S ( ϕ ) = {\hat{d}}_{0} + {\sum }_{k = 1}^{2} ( {\hat{d}}_{cos , k} cos ( ϕ ) + {\hat{d}}_{sin , k} sin ( ϕ ) ) . \qquad ( S \text{.20} )$$

(Equivalently, the control and label coefficients could be inserted into separate 2nd-order Fourier models, followed by subtracting the models.) To compute pulsatility, S(ϕ) are evaluated over a fine grid of ϕ values (for example, ϕ∈[0, 2π] in steps of 2π·0.0001), and then PI can be numerically calculated with Eq. 4 of the main text (reproduced below for convenience):

$$PI = \frac{{S ( ϕ )}_{max} - {S ( ϕ )}_{min}}{{S ( ϕ )}_{mean}} \qquad ( S \text{.21} )$$

The stability of the PI measurements can be assessed using a residuals permutation approach. The fitted data estimates Ŷ_{C} and Ŷ_{L} may be given as:

$$\begin{matrix}{\hat{Y}}_{C} = {X}_{C} \hat{c} \\ {\hat{Y}}_{L} = {X}_{L} \hat{l}\end{matrix} \qquad ( S \text{.22} )$$

and then computed the residuals \tilde{Y}_{C} and \tilde{Y}_{L} as:

$$\begin{matrix}{\tilde{Y}}_{C} = {Y}_{C} - {\hat{Y}}_{C} \\ {\tilde{Y}}_{L} = {Y}_{L} - {\hat{Y}}_{L}\end{matrix} \qquad ( S \text{.23} )$$

Then, the residuals in time (separately for labels and controls) may be randomly permuted and added back to the fitted data to create simulated data vectors Y_{C,Perm,i} and Y_{L,Perm,i}:

$$\begin{matrix}{Y}_{C , Perm , i} = {\hat{Y}}_{C} + {P}_{C , i} ( {\tilde{Y}}_{C} ) \\ {Y}_{L , Perm , i} = {\hat{Y}}_{L} + {P}_{L , i} ( {\tilde{Y}}_{L} )\end{matrix} \qquad ( S \text{.24} )$$

where P_{C,i}(·) and P_{L,i}(·) denote the permuting/shuffling operator of controls and labels, respectively, for the ith iteration. Using these simulated data vectors, PI was computed again following Eqs. S.18-S.21. This procedure was repeated over 1000 iterations, and the resulting distribution of PI values were used to compute 95% confidence intervals.

### S3 Impact of Measurement Noise on Pulsatility

This section assesses the bias and SNR of the PI measurement under measurement noise (i.e., noisy control and label data points). The theoretical relationship between measurement noise and the distributions of the estimated difference coefficients \hat{d}₀, \hat{d}_{cos,1}, \hat{d}_{sin,1}, \hat{d}_{cos,2} and \hat{d}_{sin,2} (which were estimated in Eq. S.19) may be derived. The impact of noise on PI is then assessed via simulations and theoretical approximations of the SNR of PI.

### S3.1 Theory

### S3.1.1 SNR and Bias of Pulsatility Index

We first approach this problem by assuming ground truth 2nd-order Fourier coefficients for the control, label and difference signals as follows:

$$\begin{matrix}Control coefficient c \\ Label coefficients l \\ Difference coefficients d = c - 1\end{matrix} \qquad ( S \text{.25} )$$

respectively. In a VSASL scanning experiment, control and label data points may be acquired at cardiac phases of ϕ_{C} and ϕ_{L} (defined in Eq. S.14). Assuming a 2nd-order Fourier model for the present data, the design matrices described in Eq. S.15 may be used to express the present data points as:

$$\begin{matrix}{Y}_{C} = {X}_{C} c + {ò}_{C} {ò}_{C} ▯ N ( 0 , {σ}_{C}^{2} I ) \\ {Y}_{L} = {X}_{L} l + {ò}_{L} {ò}_{L} ▯ N ( 0 , {σ}_{L}^{2} I )\end{matrix} \qquad ( S \text{.26} )$$

where X_{C} and X_{L} are defined as in Eq. S.15. The terms Ò_{C} and Ò_{L} represent measurement noise, which are assumed to be independent and identically distributed (i.i.d.) normal random variables with variances

$${σ}_{C}^{2} and {σ}_{L}^{2} ,$$

respectively. (Normality of noise in MRI magnitude images is a reasonable approximation when the SNR of a given voxel is sufficiently high. The noise begins to resemble a normal distribution at SNRs as low as ˜3. The control and label data of this study comfortably clear this regime, with empirical voxel-wise SNRs on the order of ˜50 for cortical gray matter voxels.)

By substituting these expressions of Y_{C} and Y_{L} into least squares estimates of ĉ and Î described in Eq. S.18:

$$\begin{matrix}c = c + {( {X}_{C}^{T} {X}_{C} )}^{- 1} {X}_{C}^{T} {ò}_{C} \\ l = l + {( {X}_{L}^{T} {X}_{L} )}^{- 1} {X}_{L}^{T} {ò}_{L}\end{matrix} \qquad ( S \text{.27} )$$

These expressions can then be substituted into Eq. S.19 to obtain an estimate of difference coefficients \hat{d} as:

$$\hat{d} = d + {( {X}_{C}^{T} {X}_{C} )}^{- 1} {X}_{C}^{T} {ò}_{C} - {( {X}_{L}^{T} {X}_{L} )}^{- 1} {X}_{L}^{T} {ò}_{L} . \qquad ( S \text{.28} )$$

In a realistic experiment, the entries of ϕ_{C} (and ϕ_{L}) have values in [0, 2π] that are unevenly spaced across the interval. However, when a sufficient number of measurements (e.g., N=72 as in the present experiments) may be available, approximating ϕ_{C} (and ϕ_{L}) as vectors with uniformly spaced entries has negligible impact on the ĉ (and \hat{l}) estimation (results not shown). This evenly-spaced approximation is:

$${ϕ}_{C} \approx {ϕ}_{L} \approx ϕ ( N \times 1 ) \qquad ( S \text{.29} )$$

whose entries are given by:

$${ϕ}_{i} = \frac{{2}_{π} ( i - 1 )}{N} for i = 1 , … , N \qquad ( S \text{.30} )$$

Consequently, the design matrices are also approximated as:

$${X}_{C} \approx {X}_{L} \approx X = [ \begin{matrix}\vert & \vert & \vert & \vert & \vert \\ 1 & sin ( ϕ ) & cos ( ϕ ) & sin ( 2 ϕ ) & cos ( 2 ϕ ) \\ \vert & \vert & \vert & \vert & \vert\end{matrix} ] \qquad ( S \text{.31} )$$

and Eq. S.28 simplifies to:

$$\hat{d} = d + {( {X}^{T} X )}^{- 1} {X}^{T} {{o}^{`}}_{D} \qquad ( S \text{.32} )$$

where

$${{o}^{`}}_{C} - {{o}^{`}}_{L} = {{o}^{`}}_{D} N ( 0 , {σ}_{D}^{2} I )$$

$$and$$

$${σ}_{D}^{2} = {σ}_{C}^{2} + {σ}_{L}^{2} .$$

By assessing the expected value and covariance, \hat{d} can be described as a normally distributed random vector:

$$\hat{d} ∼ N ( d , C ) \qquad ( S \text{.33} )$$

$$where$$

$$( S \text{.34} )$$

$$C = [ \begin{matrix}\frac{{σ}_{D}^{2}}{N} & 0 & 0 & 0 & 0 \\ 0 & \frac{2 {σ}_{D}^{2}}{N} & 0 & 0 & 0 \\ 0 & 0 & \frac{2 {σ}_{D}^{2}}{N} & 0 & 0 \\ 0 & 0 & 0 & \frac{2 {σ}_{D}^{2}}{N} & 0 \\ 0 & 0 & 0 & 0 & \frac{2 {σ}_{D}^{2}}{N}\end{matrix} ] = \frac{2 {σ}_{D}^{2}}{N} \cdot ( I - [ \begin{matrix}1 & 0 & 0 & 0 & 0 \\ 0 & 0 & 0 & 0 & 0 \\ 0 & 0 & 0 & 0 & 0 \\ 0 & 0 & 0 & 0 & 0 \\ 0 & 0 & 0 & 0 & 0\end{matrix} ] )$$

with off-diagonal terms equal to 0 due to the orthogonality of the evenly-spaced 2nd-order Fourier regressors. This expression explicitly connects the variance of measurement noise

$$( {σ}_{D}^{2} = {σ}_{C}^{2} + {σ}_{L}^{2} )$$

to the stability of the difference (ASL signal) coefficient estimates \hat{d}.

By inserting the coefficients into Ŝ(ϕ) (Eq. S.20) and using arguments from SI Section S1 to neglect second-order terms, the pulsatility index estimate PI is then given by:

$$PI = \frac{2 \sqrt{{\hat{d}}_{cos , 1}^{2} + {\hat{d}}_{sin , 1}^{2}}}{{\hat{d}}_{0}} . \qquad ( S \text{.35} )$$

The SNR of PI is then defined as

$$SNR ( PI ) = \frac{E [ PI ]}{Std ( PI )} \qquad ( S \text{.36} )$$

where E[PI] is the expected value and Std(PI)=\sqrt{Var(PI)}. The bias of PI is defined as:

$$bias ( PI ) = E [ PI ] - PI . \qquad ( S \text{.37} )$$

### S3.1.2 Approximation of SNR

When examining PI (Eq. S.35), its numerator can be identified as a Rician random variable which may be denoted as \hat{ν}:

$$\hat{v} = 2 \sqrt{{\hat{d}}_{cos , 1}^{2} + {\hat{d}}_{sin , 1}^{2}} □ Rice ( v , q ) \qquad ( S \text{.38} )$$

with

$$v = 2 \sqrt{{d}_{cos , l}^{2} + {d}_{sin , l}^{2}} and q = 2 \sqrt{2 {σ}_{D}^{2} / N} = \sqrt{8 {σ}_{D}^{2} / N}$$

denoting the non-centrality and scale parameters of the distribution, respectively. On the other hand, its denominator can be identified as a normal random variable as described by Eq. S.34

$${\hat{d}}_{0} N ( {d}_{0} , \frac{{σ}_{D}^{2}}{N} ) . \qquad ( S \text{.39} )$$

Thus, the pulsatility estimate of

$$PI = \frac{\hat{v}}{{\hat{d}}_{0}} \qquad ( S \text{.40} )$$

is the ratio of a Rician random variable and a normal random variable. Through several simplifications, an approximation of PI may be derived that facilitates the pulsatility SNR model presented in Eq. 6 of the main text. The subsequent equations focus on the derivations, and the errors associated with each step may be examined further in the Methods and Results.

To illustrate these approximations, start with a random variable Z defined as the ratio of X and Y

$$Z = \frac{X}{Y} , \qquad ( S \text{.41} )$$

where X [symbol] Rice (μₓ,σₓ) has a non-centrality parameter μₓ and scale parameter σₓ, and Y [symbol] N (μ_{y}, σ_{y}²) has a mean μ_{y} with standard deviation σ_{y}. Provided a sufficiently high value of μₓ/σₓ, the Rician numerator X can be approximated as a normal random variable Xₙₒᵣₘₐₗ [symbol] N (μₓ, σₓ²), which yields a ratio of two normal random variables. Provided a sufficiently high value of μ_{y}/σ_{y} (which makes the denominator Y unlikely to observe negative values), the ratio of two normal random variables can be approximated as a single normal random variable with the form:

$${Z}_{approx} N ( \frac{{μ}_{x}}{{μ}_{y}} , {( \frac{\sqrt{{σ}_{x}^{2} + {σ}_{y}^{2} \cdot {( {μ}_{x} / {μ}_{y} )}^{2}}}{{μ}_{y}} )}^{2} ) . \qquad ( S \text{.42} )$$

This approximation can be applied to PI by identifying the corresponding terms

$${μ}_{x} = 2 \sqrt{{d}_{cos , 1}^{2} + {d}_{sin , 1}^{2}} , {σ}_{x} = \sqrt{8 {σ}_{D}^{2} / N} ,$$

μ_{y}=d₀ and

$${σ}_{y} = \sqrt{{σ}_{D}^{2} / N} .$$

Using the following definitions for d₀, d_{cos,1} and d_{sin,1} based on Eq. 3:

$${d}_{0} = {b}_{0} \cdot τ \cdot exp ( - \frac{τ}{{T}_{1 b}} ) \qquad ( S \text{.43} )$$

$${d}_{cos , 1} = {b}_{cos , 1} \cdot τ \cdot exp ( - \frac{τ}{{T}_{1 b}} ) \cdot sin c ( \frac{τ}{Γ} )$$

$${d}_{sin , 1} = {b}_{sin , 1} \cdot τ \cdot exp ( - \frac{τ}{{T}_{1 b}} ) \cdot sin c ( \frac{τ}{Γ} ) ,$$

we re-express those corresponding terms as functions of T:

$${μ}_{x} = 2 \sqrt{{b}_{cos , 1}^{2} + {b}_{sin , 1}^{2}} \cdot τ \cdot exp ( - τ / {T}_{1 b} ) \cdot \vert sin c ( \frac{τ}{Γ} ) \vert \qquad ( S \text{.44} )$$

$${σ}_{x} = \sqrt{8 {σ}_{D}^{2} / N} \qquad ( S \text{.45} )$$

$${μ}_{y} = {b}_{0} \cdot τ \cdot exp ( - τ / {T}_{1 b} ) \qquad ( S \text{.46} )$$

$${σ}_{y} = \sqrt{{σ}_{D}^{2} / N} . \qquad ( S \text{.47} )$$

Using the above definitions of μₓ, σₓ, μ_{y} and σ_{y}, FIG. S3 serves to qualitatively show the regimes of τ where the approximations used in Eq. S.42 (based on the ratios μₓ/σₓ and μ_{y}/σ_{y}) generally hold.

Applying Eq. S.42 yields the approximation:

$${PI}_{approx , 1} ▯ N ( PI , {( \frac{\sqrt{( 8 + {PI}^{2} ) \cdot {σ}_{D}^{2} / N}}{τ \cdot exp ( - τ / {T}_{1 b} )} )}^{2} ) , \qquad ( S \text{.48} )$$

where

$$PI = \frac{2 \sqrt{{b}_{cos , 1}^{2} + {b}_{sin , 1}^{2}}}{{b}_{0}} \cdot \vert sin c ( \frac{τ}{Γ} ) \vert$$

per Eq. 5 of the main text. Computing the SNR of PIₐₚₚᵣₒₓ (via Eq. S.36) yields:

$${SNR}_{approx , 1} = \frac{PI \cdot τ \cdot exp ( - τ / {T}_{1 b} )}{\sqrt{( 8 + {PI}^{2} ) \cdot {σ}_{D}^{2} / N}} . \qquad ( S \text{.49} )$$

By examining the \sqrt{8+PI²} factor (present in the standard deviation of PI_{approx,1} and the denominator of SNR_{approx,1}) in a plausible physiological regime, a further simplification may be made. Assuming values of b₀=1 and

$$\sqrt{{b}_{cos , 1}^{2} + {b}_{sin , 1}^{2}} = 0.5$$

(as in Section S1.2), the \sqrt{8+PI²} factor simplifies into

$$\sqrt{8 + {\vert sinc ( \frac{τ}{Γ} ) \vert}^{2}} .$$

Note that the

$${\vert sinc ( \frac{τ}{Γ} ) \vert}^{2}$$

term has a maximum value of 1 (at τ=0 s) that quickly approaches 0 with increasing τ due to its envelope of (Γ/πτ)², thus representing a negligible contribution to the overall value of the factor. For example, assuming Γ=1 s, neglecting

$${\vert sinc ( \frac{τ}{Γ} ) \vert}^{2}$$

at values of τ=0/0.5/1.5 s produces errors of 0.057/0.038/0.013 when expressed as a fraction of

$$\sqrt{8 + {\vert sinc ( \frac{τ}{Γ} ) \vert}^{2}} ,$$

respectively.

As empirical support, one can also note that the measured values of PI were all on the order of 1 or less (FIGS. 3 and 6). Thus, for a reasonable physiological regime, further approximate may be made \sqrt{8+PI²}≈\sqrt{8}, which yields another approximation of PI:

$${PI}_{approx , 2} ▯ N ( PI , {( \frac{\sqrt{( 8 {σ}_{D}^{2} / N )}}{τ \cdot exp ( - τ / {T}_{1 b} )} )}^{2} ) \qquad ( S \text{.50} )$$

and analytical model of the SNR:

$${SNR}_{approx , 2} = \frac{PI \cdot τ \cdot exp ( - τ / {T}_{1 b} )}{\sqrt{8 {σ}_{D}^{2} / N}} . \qquad ( S \text{.51} )$$

Note that by expanding out the

$$PI = \frac{2 \sqrt{{b}_{cos , 1}^{2} + {b}_{sin , 1}^{2}}}{{b}_{0}} \cdot \vert sinc ( \frac{τ}{Γ} ) \vert$$

term, this final expression yields the SNR model presented in Eq. 6 of the main text (reproduced below):

$$SNR ( τ ) ∝ τ \cdot exp ( - \frac{τ}{{T}_{1 b}} ) \cdot \vert sinc ( \frac{τ}{T} ) \vert . \qquad ( S \text{.52} )$$

Thus, Eq. S.51 is also optimized at τₒₚₜ, given by Eq. 7 of the main text.

### S3.2 Methods

Using the theory derived in Section S3.1.1, simulations may be used to assess the impact of noise on the bias and SNR of PI measurements, particularly as a function of τ. To do so, (1) assume a noiseless reference PI(τ) curve, (2) add measurement noise, (3) compute distributions of PI(τ) at every τ value, and (4) assess the bias and SNR of PI(τ) as a function of τ.

We begin by assuming reference CBF(t) coefficients b₀, b_{cos,1}, b_{sin,1}, b_{cos,2} and b_{sin,2}. Assume similar values as in Section S1 (the sinc model error simulations) by using b₀=1, splitting the

$$\sqrt{{b}_{cos , 1}^{2} + {b}_{sin , 1}^{2}} = 0.5$$

constraint into b_{cos,1}=b_{sin,1}=\sqrt{0.125}, and setting b_{cos,2}=b_{sin,2}=0 to neglect the 2nd-order component for simplicity.

Then, over a grid of τ∈[0.001, 2.000] s in steps of 0.001 s, the corresponding S(t) coefficients d=[d₀ d_{cos,1} d_{sin,1} d_{cos,2} d_{sin,2}]^{T} are computed for every value of τ based on Eq. 3 as:

$$\begin{matrix}{d}_{0} = {b}_{0} \cdot τ \cdot exp ( - \frac{τ}{{T}_{1 b}} ) \\ {d}_{cos , k} = {b}_{cos , k} \cdot τ \cdot exp ( - \frac{τ}{{T}_{1 b}} ) \cdot sinc ( \frac{k τ}{Γ} ) \\ {d}_{sin , k} = {b}_{sin , k} \cdot τ \cdot exp ( - \frac{τ}{{T}_{1 b}} ) \cdot sinc ( \frac{k τ}{Γ} )\end{matrix} \qquad ( S \text{.53} )$$

using a cardiac period Γ=1.0 s, and T_{1b}=1.6 s. Once computed, d₀, d_{cos,1}, d_{sin,1}, d_{cos,2} and d_{sin,2} are inserted into the S(ϕ) 2nd-order Fourier model (Eq. S.20) and then PI is computed (Eq. S.21). For example, at τ=500 ms, the coefficients are computed as d₀=0.366, d_{cos,1}=d_{sin,1}=0.0825 and d_{cos,2}=d_{sin,2}=0 (or equivalently, d=[0.366 0.0825 0.0825 0.000 0.000]^{T}), which produce a pulsatility value of PI=0.636. By repeating this procedure for every t value, the ground truth (reference) PI(τ) curve is obtained.

Next, noise may be added to the simulations and compute PI(τ). In order to add a realistic amount of noise, the in vivo GM ROI τ=500 ms scan analysis results were used to choose an appropriate value of

$${σ}_{D}^{2} .$$

### First,

$${σ}_{C}^{2} and {σ}_{L}^{2}$$

were estimated as the variance of the in vivo control and label fit residuals (Eq. S.23) at τ=500 ms, and then

$${σ}_{D}^{2}$$

was computed as

$${σ}_{D}^{2} = {σ}_{C}^{2} + {σ}_{L}^{2} .$$

Then, the ratio of σ_{D}/d₀ was estimated to be ≈0.10 (on average across subjects). Since d₀=0.366 at τ=500 ms in these noise simulations, σ_{D}=0.0366 may be chosen to produce an equivalent ratio of σ_{D}/d₀. This value of σ_{D}=0.0366 (i.e., noise level) was then kept constant across all values of τ, consistent with the empirical observation that the residuals at τ=750/1000/1250/1500 ms were comparable in magnitude to the residuals at τ=500 ms.

The number of measurements was set at N=72. The covariance matrix C was computed using Eq. S.34, and then 10⁵ realizations of \hat{d} were simulated following \hat{d} [symbol] N (d, C) (Eq. S.33). PI may be computed for each realization (Eq. S.21) to produce a distribution of PI values at every value of τ.

We then evaluated PI based on its SNR (Eq. S.36) and bias (Eq. S.37) at every value of τ, with the SNR curve then numerically assessed for an optimal value of τ:

$${τ}^{*} = {arg max}_{τ} SNR ( τ ) . \qquad ( S \text{.54} )$$

The analytical expressions of SNR_{approx,1} (Eq. S.49) and SNR_{approx,2} (Eq. S.51) were also evaluated at every value of τ, and similarly assessed for their optimal values as well:

$${τ}_{approx , 1}^{*} = {arg max}_{τ} {SNR}_{approx , 1} ( τ ) \qquad ( S \text{.55} )$$

$${τ}_{opt} = {τ}_{approx , 2}^{*} = {arg max}_{τ} {SNR}_{approx , 2} ( τ ) . \qquad ( S \text{.56} )$$

### S3.3 Results

Diagram (a) of FIG. S4 shows generally good agreement between the noiseless reference curve PI and the expected value E[PI], with little spread in PI as indicated by the shaded areas. However, PI becomes less accurate as r approaches 0s and the noise begins to dominate. While both the numerator and denominator of the calculation are affected by noise, a noisy denominator \hat{d}₀ can be particularly problematic for the PI calculation, as individual realizations of \hat{d}₀ can approach 0 (or even cross into negatives) and make the PI quotient approach infinity. As a result, PI can become unstable in very low-τ regimes, which is not surprising due to the low mean signal.

In diagram (b) of FIG. S4, the asymptotic behavior of the bias(PI) and Std(PI) curves for τ→0 reflects the aforementioned stability issues. Another defining feature is the spike in bias around τ=Γ (and τ=2Γ). Here, the reference PI is 0, but PI values remain strictly positive due to the Sₘₐₓ−Sₘᵢₙ numerator of Eq. 4, resulting in the positive bias. However, the bias is otherwise negligible for most values of τ, including regimes around τₒₚₜ.

Diagram (c) of FIG. S4 shows the SNR curve (Eq. S.36) alongside SNR_{approx,1} (Eq. S.49) and SNR_{approx,2} (Eq. S.51), demonstrating excellent agreement in their overall shapes. Furthermore, their respective maxima occur at very similar values of τ (represented by the vertical lines). Of note, the difference between SNR(τₒₚₜ) and SNR(τ*) is less than 0.1% (18.92 vs 18.91), showing that τₒₚₜ indeed approximately maximizes SNR. Altogether, the agreement in curve shapes and negligible difference between SNR(τₒₚₜ) and SNR(τ*) supports the use of the simple theoretical model (Eq. 6) to describe the τ-dependence of SNR and guide the selection of an optimal t value.

Examining the curves more closely, SNR_{approx,1} is nearly identical to SNR except around τ=Γ and τ=2Γ. The difference in these regimes is expected, as the assumption of normality of the PI numerator, built into Eq. S.42 (and thus into SNR_{approx,1}), diverges from its Rician nature in these low-SNR regimes. FIG. S5 serves as a supporting figure by comparing the simulated PI distribution (blue histogram) and the analytical PDF of PI_{approx,1} (solid line), which approximates the PI distribution poorly at τ=Γ and τ=2Γ, but otherwise shows excellent agreement for the τ values shown. Examining SNR_{approx,2}, its curve (dotted line) is nearly identical to SNR_{approx,1} but is observed to be slightly higher (noticeable in the regime around 0.2 s to 0.6 s) due to the PI² term that was neglected in the derivation step from Eq. S.49 to Eq. S.51.

FIG. S3 qualitatively shows the regimes of τ where the approximations used in Eq. S.42 may be expected to hold.

### S4 T1 Structural Scan Parameters

Two T1 structural scan configurations were used with similar parameters and both based on a Magnetization-Prepared Rapid Acquisition Gradient Echo (MPRAGE) sequence. The main difference was the acceleration factor, with one using 4×-acceleration for shorter scan time, and the other using 2×-acceleration to maintain higher SNR for a separate project. The parameters for the former configuration (with the latter configuration noted in parentheses wherever different) were: resolution=0.9375 mm isotropic (1 mm isotropic), matrix size=176×256×256, 176 slices in the sagittal orientation, flip angle=8 degrees, TR=2300 ms (2500 ms), TE=2.29 ms (2.88 ms), inversion time=900 ms (1060 ms), GRAPPA=4×(2×) acceleration in the A-P phase-encode direction, acquisition time=3:08 (7:30).

### S5 Iterative Denoising

This section provides supporting details for the iterative denoising method referenced in the main text Section B.3, which was adapted from Power et al. with a few modifications.

The algorithm involves (1) regressing out nuisance variance and also (2) censoring outlier volumes based on the regression residuals. First, nuisance regressors were constructed (motion timecourses, motion first-derivative timecourses, and first- and second-order low-frequency drift). To preserve cardiac-driven fluctuations, 2nd-order Fourier regressors of controls and labels were included. These regressors were constructed by starting with X_{C} and X_{L} (derived in Eq. S.15 in SI Section S2) and interleaving with 0's to account for the alternating control/label ASL acquisition scheme. For example, 0's were inserted into the X_{C} regressors for every row corresponding to a label data point (and vice versa for the X_{L} regressors at control data points). Diagram (a) of FIG. S6 shows how they are interleaved with 0's and then concatenated alongside the nuisance regressors. Diagram (b) of FIG. S6 shows X_{C} and X_{L} separately and sorted in cardiac phase ϕ order to better illustrate the shape of these regressors.

The initial temporal censoring mask included volumes with framewise displacement (FD) exceeding 0.75 mm (with FD computed by others), and volumes with a cardiac period Γ more than 3 scaled median absolute deviations away from the median of the Γ timecourse. The outliers in Γ often corresponded to index finger motion, which distorted the PPG trace and caused unreliable retrospective gating. These volumes were censored before the first iteration of the algorithm.

The nuisance and Fourier regressors were both input into the iterative denoising algorithm, and additional timepoints were censored with each iteration based on DVARS and SD metrics. The algorithm was ended when no new timepoints were censored for a given iteration. Then the nuisance regressors were orthogonalized with respect to the Fourier regressors, and the orthogonalized nuisance fits were subtracted out.

### S7 Figures

FIG. S1: Evaluating Sinc Approximation Error: (a) Best case fit of Eq. 5 to the exact PI(τ) curve. The curves are nearly identical, with negligible absolute error (<0.01) over all values of τ and R²=1.0000. (b) Worst case fit of Eq. 5 to the exact PI(τ) curve. Even in this worst case, the agreement between curves is excellent, with minimal absolute error (<0.05) over all values of τ and R²=0.9964. (c) Image of the absolute error across all values of ϕᵣₑₗ∈[0, 2π].

FIG. S2: Comparison of the τ-stepping results when using 2nd-vs 1st-order Fourier models to describe S(ϕ). Shown in (a) and (b) are results from two representative subjects (4 and 5). Plotted in blue (L6 and L7) are the τ-stepping results using a 2nd-order Fourier model (identical to those shown in FIG. 8 of the main text). Plotted in orange (L8 and L9) are the PI values when using a 1st-order Fourier model. The insets show comparisons of the S(ϕ) curves for each approach. Shown in (c) is a comparison of A (the fitting parameter of the sinc model). Shown in (d) is a comparison of the R² of the sinc model fits.

FIG. S3: A supporting figure for qualitatively understanding the regimes of τ where the approximations used in Eq. S.42 generally hold. The expressions of μₓ, σₓ, μ_{y}, and σ_{y} were evaluated using Eq. S.44, with values of b₀=1, b_{cos,1}=b_{sin,1}=\sqrt{0.125}, Γ=1 s, σ_{D}=0.0366 and N=72 assumed as in Section S3.2. The blue regions (L14) indicate where μₓ/σₓ is less than a threshold of 5, where the normal approximation of the Rician numerator of PI becomes relatively poor. The orange regions (L15) indicate where μ_{y}/σ_{y} is less than a threshold of 10, where the approximation of the ratio of two normal random variables as a single normal random variable becomes relatively poor. The green regions (L16) indicate the regimes where both thresholds are exceeded and the approximations hold relatively well. These green areas are consistent with the regimes in diagram (c) of FIG. S4 showing excellent agreement between SNR and SNR_{approx,1}.

FIG. S4: Noise simulations for PI. (a) Reference PI curve (solid line L32), expected value of PI (dashed line L34), and the spread of PI (shaded regions corresponding to 68% and 95% confidence intervals). (b) The bias (orange) and standard deviation (cyan) of PI as a function of τ. (c) The SNR of the PI measurement (solid line L22) and approximations SNR_{approx,1} (Eq. S.49, dashed line L24) and SNR_{approx,2} (Eq. S.51, dash-dotted line L28). Also indicated are τ*,

$${τ}_{approx , 1}^{*}$$

and τₒₚₜ (vertical lines), i.e., the τ values where their respective maxima occur. The plotted points indicate the SNR evaluated at those τ values.

FIG. S5: This is a supporting figure for understanding the SNR curves shown in diagram (c) of FIG. S4. Shown are the simulated distribution of PI (blue histogram) compared to the analytical PDFs of PI_{approx,1} (solid line) and PI_{approx,2} (dotted line), for a range of selected τ values in steps of 0.250 s (except the first step at 0.100 s). Also indicated are the reference PI value (vertical solid line) and E[PI] (vertical dashed line). At each value of τ, the texts show the values involved in computing the SNR. The histogram and its analytical approximations (PI_{approx,1} and PI_{approx,2}) generally agree well, except at τ=Γ and 2Γ, where the built-in normal approximation of the Rician numerator of PI no longer holds.

FIG. S6: Illustration of the 2nd-order Fourier models in the iterative denoising algorithm. (a) The overall design matrix used in the iterative denoising algorithm of Section B.3 of the main text. The 2nd-order Fourier matrices, representing separate models for controls and labels are sorted in time according to the temporal acquisition order of the control and label volumes and interleaved with O's according to the label/control alternation. They are then concatenated alongside the nuisance regressors (drift, motion and motion derivatives) and input into the iterative denoising algorithm. (b) The 2nd-order Fourier design matrices (constructed as in Eq. S.15) separated out and sorted in cardiac phase ϕ order for visualization.

### S8.

### S8.1 Theory

The control-tag subtraction signal intensity (denoted S) from a VSASL scan is proportional to the product of cerebral blood flow (CBF) and bolus duration τ. In the case of time-varying, pulsatile flow, the product may be replaced with the following convolution:

$$S ( t ) ∝ CBF ( t ) * r e c t ( \frac{t}{τ} ) \qquad ( S 8.1 )$$

S(t) may be considered a smoothed version of CBF(t), with the degree of smoothing controlled by τ. To examine cardiac-driven pulsatility, assume a simple model for CBF(t), a pure sinusoid with oscillation amplitude A, mean B, and cardiac period T:

$$CBF ( t ) = A sin ( \frac{2 π t}{T} ) + B \qquad ( S 8.2 )$$

Inserting this expression into Eq. S8.1 yields:

$$S ( t ) ∝ \frac{AT}{π} sin ( \frac{π τ}{T} ) sin ( \frac{2 π t}{T} ) + B τ = A τ \cdot sinc ( \frac{τ}{T} ) sin ( \frac{2 π t}{T} ) + B τ \qquad ( S 8.3 )$$

where

$$sinc ( x ) = \frac{sin ( π x )}{π x} .$$

To characterize pulsatility, a previously defined pulsatility index (PI) may be used. For a pulsatile signal y(t):

$${PI}_{y ( t )} = \frac{{y ( t )}_{max} - {y ( t )}_{min}}{{y ( t )}_{mean}} \qquad ( S 8.4 )$$

Applying this to CBF(t) yields:

$${PI}_{CBF} = \frac{2 A}{B} \qquad ( S 8.5 )$$

and to S(t) yields:

$${PI}_{S} = ( \frac{2 A}{B} ) \vert sinc ( \frac{τ}{T} ) \vert = {PI}_{CBF} \cdot \vert sinc ( \frac{τ}{T} ) \vert \qquad ( S 8.6 )$$

To account for heart rate variability, a modified version of Eq. S8.6 may be used.

$${PI}_{S} = {PI}_{CBF} \cdot κ ( τ ) \qquad ( S 8.7 )$$

where κ(τ) is the weighted average of

$$\vert sinc ( \frac{τ}{T} ) \vert$$

functions:

$$κ ( τ ) = {\sum }_{i} p ( {T}_{i} ) \vert sin c ( \frac{τ}{{T}_{i}} ) \vert \qquad ( S 8.8 )$$

and p(Tᵢ) is the probability of observing Tᵢ for the subject.

Given Eq. S8.7, PI_{CBF} can subsequently be estimated from a measurement of PIs.

### S8.2 Methods

### Data Acquisition

Three healthy subjects (3 males, aged 23-45 years) were scanned under UCSD IRB approval, with informed consent obtained from all participants. MRI scans were acquired on a Siemens Prisma 3.0 T system with a 32ch head coil. Sessions consisted of (1) VSASL scans with τ from 250 ms to ˜1.5× the subject's median Tin steps of 250 ms, and (2) a T1-weighted MPRAGE. All VSASL scans used an identical 3D GRASE readout (resolution=4×4×6 mm³, matrix size=56×56×24, single-shot, TE=15.42 ms, GRAPPA acceleration 2×2 in PE×Slice). See Table 1 for VS labeling and timing parameters. All VSASL scans were acquired while recording the subject's cardiac cycle using a pulse oximeter finger cuff.

### Data Processing

Motion correction of VSASL data and tissue segmentation of the T1 scan were performed using FSL. The GM partial volume map was thresholded at 0.8 and resampled to the VSASL resolution to create the GM mask. Areas of bulk CSF artifact were manually excluded. Using this final mask, mean GM signal intensity was extracted from each VSASL timepoint.

### Pulsatility Analysis

Pulse oximeter signals were smoothed, followed by peak detection. Following previously described approaches, a fractional cardiac phase was assigned to each volume, data were fit to a 2^{nd}-order Fourier function, and PI_{S} was computed for each VSASL scan via Eq. S8.4.

To assess agreement with the present model, κ(τ) was calculated via Eq. S8.8, with p(Tᵢ) estimated from the distribution of Ts from the pulse oximeter signal. Then, each subject's PI_{S} vs τ data were fit to Eq. S8.7. To assess repeatability, each subject's τ=500 ms scans were also split into two halves and PI_{CBF} was estimated from PI_{S} with Eq. S8.7 for each half. Percent difference in PI_{CBF} between each half was then computed.

### S8.3 Results

FIG. S8.1 shows PI_{S} vs τ for each subject and the fit of their data to Eq. S8.7. The local minimum of PI_{S} around τ=T is an important validation that measures cardiac-driven pulsatility, since from Eq. S8.3 little fluctuation in S may be expected when τ is exactly one cardiac period.

FIG. S8.2 shows the estimated PI_{CBF} values from the two halves of the τ=500 ms scans to test repeatability. The results show good repeatability with this configuration, with a mean percent difference of 6.17% between successive halves.

### S8.4 Discussion

The results demonstrate measurement of CBF pulsatility within the microvasculature by exploiting the unique microvascular specificity of VSASL.

Reflecting the effect of the

$$sin ( π \frac{τ}{T} )$$

term in Eq. S8.3, the fluctuation amplitude of S is maximized with τ=T/2, making it optimal for measuring PI_{S}. For a typical T near 1s, τ=500 ms satisfies this criterion.

The microvascular pulsatility may be presumably driven by signal changes slightly upstream, supported by Franklin et al. showing cardiac-driven signal changes up to 36% in the VS-labeled arterial pool. Notably, prior work by others reported lower PI values in the microvasculature than the current work; however, the prior work measured PI on flow velocity, as opposed to CBF, which may explain this discrepancy. The current technique may consider more physiologically realistic waveforms for CBF(t), and may include refined treatment of heart-rate variability by incorporating it earlier in the theory into CBF(t). One or both modifications may facilitate better modeling of downstream effects on pulsatility measurements.

### S8.5 Conclusion

The document shows a repeatable approach of using VSASL to measure CBF pulsatility in the microvasculature.

FIG. S7. PI_{S} values plotted vs t and fitted to Eq. S8.7 for the three subjects. The primary observation is that for each subject, there is a local minimum of PIs in the vicinity of T, and higher PI_{S} elsewhere, consistent with the proposed model.

FIG. S8. Results of the repeatability experiment, showing the estimated PI_{CBF} values for the two halves of the τ=500 ms scans. The scan of τ=500 ms was chosen since τ is around half the cardiac period for most subjects. PI_{CBF} was estimated from PI_{S} using Eq. S8.7 with κ(τ) based on the T's from the given scan half. Across three subjects, the mean percentage difference between halves was 6.17%.

| | | | | | | | # | Scan | | | |
|---|---|---|---|---|---|---|---|---|---|---|---|
| Scan | Tₛₐₜ | τ | PLD | BGS1 | BGS2 | TR | T/C | Time | Subject | Subject | Subject |
| Number | (ms) | (ms) | (ms) | (ms) | (ms) | (ms) | Pairs | (min) | 1 | 2 | 3 |
| 1 | 1600 | 250 | 77 | 15 | — | 2300 | 98 | 7.5 | x | x | x |
| 2 | 1600 | 500 | 20 | 140 | — | 2500 | 120 | 10 | x | x | x |
| 3 | 1600 | 750 | 74 | 28 | 628 | 2800 | 80 | 7.5 | x | x | x |
| 4 | 1600 | 1000 | 41 | 15 | 760 | 3000 | 74 | 7.5 | x | x | x |
| 5 | 1600 | 1250 | 63 | 83 | 962 | 3300 | 68 | 7.5 | x | — | x |
| 6 | 1600 | 1500 | 105 | 315 | 1260 | 3600 | 64 | 7.5 | x | — | x |

Table S8.1. Parameters for the VSASL scans. Tₛₐₜ was kept constant at 1600 ms, while the PLD and BGS timings were varied to give good background suppression for each τ. VS modules used DRHS pulse trains with V_{cut}=2 cm/s. BGS timings are given as time after the VS labeling module.

### S9. Retroactive Cardiac Gating

FIG. S9 illustrates retroactive cardiac gating according to some embodiments of the present document. Diagram (1) illustrates tag and control images acquired in a VSASL scan. Diagrams (2) and (3) show, using retrospective gating, VSASL signals acquired over multiple cardiac cycles may be retroactively gated into one cardiac cycle to provide signal intensity information over the cardiac cycle. By doing this voxelwise, perfusion weighted images across cardiac cycle may be obtained. The signal intensity information may be presented in the form of a signal intensity curve as shown in diagram (3) to conveniently show the maximum, minimum, and mean signal intensities. Pulsatility indexes may be determined to characterize pulsatility.

### EXAMPLES

The following examples are illustrative of several embodiments in accordance with the present technology. Other exemplary embodiments of the present technology may be presented prior to the following listed examples, or after the following listed examples.
Example 1. A method, comprising:
   magnetically labelling blood flow in a target area of a subject using a velocity-selective arterial spin labeling (VSASL) technique with a cutoff velocity by performing operations including:
      applying a first velocity-selective (VS) pulse sequence with the cutoff velocity to mark a leading edge of a blood bolus; and
      applying a second VS pulse sequence with the cutoff velocity to mark a trailing edge of the blood bolus;
   acquiring VSASL signals of the blood bolus for voxels corresponding to the target area;
   for each of the voxels,
      obtaining signal intensity information over a cardiac cycle by performing retroactive cardiac gating on the acquired VSASL signals corresponding to the voxel; and
      determining a pulsatility index for the voxel based on the signal intensity information; and
   generating a voxel-wise pulsatility index map for the target area using the pulsatility indexes of the voxels.
Example 2. The method of any one or more examples as disclosed herein, wherein the application of the second VS pulse sequence is separated from the application of the first VS pulse sequence by a bolus duration.
Example 3. The method of any one or more examples as disclosed herein, further comprising selecting the bolus duration based on a cardiac period of the subject.
Example 4. The method of any one or more examples as disclosed herein, wherein the bolus duration is half of the cardiac period of the subject.
Example 5. The method of any one or more examples as disclosed herein, further comprising: selecting the cutoff velocity based on a dimension of blood vessels in the target area or a location of the target area along an arterial network of the subject.
Example 6. The method of any one or more examples as disclosed herein, wherein determining the pulsatility index for the voxel based on the signal intensity information comprises:
   determining a maximum signal intensity, a minimum signal intensity, and a mean signal intensity of the signal intensity information of the voxel; and
   determining the pulsatility index of the voxel based on the maximum signal intensity, the minimum signal intensity, and the mean signal intensity.
Example 7. The method of any one or more examples as disclosed herein, wherein acquiring VSASL signals of the blood bolus for the voxels comprises:
   obtaining raw VSASL signals by performing a VSASL scan over the target area; and
   obtaining the VSASL signals for the voxels by performing at least one of co-registration or motion correction of the raw VSASL signals.
Example 8. The method of any one or more examples as disclosed herein, wherein the VSASL scan follows the application of the second VS pulse sequence by a post-labelling delay (PLD).
Example 9. The method of any one or more examples as disclosed herein, wherein the target area is in the brain of the subject, the method further comprising: applying at least one of a spectrally-selective fat-saturation module or an inferior saturation module within the PLD.
Example 10. The method of any one or more examples as disclosed herein, wherein performing retroactive cardiac gating on VSASL signals corresponding to the voxel comprises: assigning a cardiac phase to each of the VSASL signals.
Example 11. The method of any one or more examples as disclosed herein, wherein at least one of the first VS pulse sequence or the second VS pulse sequence comprises an eight-segment Bo/B+insensitive rotation (BIR-8) train.
Example 12. A magnetic resonance imaging (MRI) system, comprising:
   a scanner comprising a magnet;
   gradient coils; and
   at least one processor, wherein the at least one processor is configured to perform a process including:
      magnetically labelling blood flow in a target area of a subject using a velocity-selective arterial spin labeling (VSASL) technique with a cutoff velocity by performing operations including:
         applying a first velocity-selective (VS) pulse sequence with the cutoff velocity to mark a leading edge of a blood bolus; and
         applying a second VS pulse sequence with the cutoff velocity to mark a trailing edge of the blood bolus;
      acquiring VSASL signals of the blood bolus for voxels corresponding to the target area;
      for each of the voxels,
         obtaining signal intensity information over a cardiac cycle by performing retroactive cardiac gating on VSASL signals corresponding to the voxel; and
         determining a pulsatility index for the voxel based on the signal intensity information; and
      generating a voxel-wise pulsatility index map for the target area using the pulsatility indexes of the voxels
Example 13. The MRI system of any one or more examples as disclosed herein, wherein a magnetic field strength of the MRI system is lower than 7 Tesla.
Example 14. The MRI system of any one or more examples as disclosed herein, wherein the application of the second VS pulse sequence is separated from the application of the first VS pulse sequence by a bolus duration.
Example 15. The MRI system of any one or more examples as disclosed herein, wherein the bolus duration relates to a cardiac period of the subject.
Example 16. The MRI system of any one or more examples as disclosed herein, wherein the cutoff velocity based on a dimension of blood vessels in the target area or a location of the target area along an arterial network of the subject.
Example 17. The MRI system of any one or more examples as disclosed herein, wherein determining the pulsatility index for the voxel based on the signal intensity information comprises:
   determining a maximum signal intensity, a minimum signal intensity, and a mean signal intensity of the signal intensity information of the voxel; and
   determining the pulsatility index of the voxel based on the maximum signal intensity, the minimum signal intensity, and the mean signal intensity.
Example 18. The MRI system of any one or more examples as disclosed herein, wherein the target area comprises the brain, a lung, a kidney, or the liver of the subject.
Example 19. The MRI system of any one or more examples as disclosed herein, wherein performing retroactive cardiac gating on VSASL signals corresponding to the voxel comprises: assigning a cardiac phase to each of the VSASL signals.
Example 20. One or more computer readable media having processor-executable code, upon execution by one or more processors, causing the one or more processors to perform a process including:
   magnetically labelling blood flow in a target area of a subject using a velocity-selective arterial spin labeling (VSASL) technique with a cutoff velocity by performing operations including:
      applying a first velocity-selective (VS) pulse sequence with the cutoff velocity to mark a leading edge of a blood bolus; and
      applying a second VS pulse sequence with the cutoff velocity to mark a trailing edge of the blood bolus;
   acquiring VSASL signals of the blood bolus for voxels corresponding to the target area;
   for each of the voxels,
      obtaining signal intensity information over a cardiac cycle by performing retroactive cardiac gating on VSASL signals corresponding to the voxel; and
      determining a pulsatility index for the voxel based on the signal intensity information; and
   generating a voxel-wise pulsatility index map for the target area using the pulsatility indexes of the voxels.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Various embodiments described herein are described in the general context of methods or processes, which may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), Blu-ray Discs, etc. Therefore, the computer-readable media described in the present application include non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

For example, one aspect of the disclosed embodiments relates to a computer program product that is embodied on a non-transitory computer readable medium. The computer program product includes program code for carrying out any one or and/or all of the operations of the disclosed embodiments.

Only a few implementations and examples are described and other implementations, enhancements and variations can be made based on what is described and illustrated in this patent document.

I/We claim:

1. A method, comprising:
magnetically labelling blood flow in a target area of a subject using a velocity-selective arterial spin labeling (VSASL) technique with a cutoff velocity by performing operations including:
applying a first velocity-selective (VS) pulse sequence with the cutoff velocity to mark a leading edge of a blood bolus; and
applying a second VS pulse sequence with the cutoff velocity to mark a trailing edge of the blood bolus;
acquiring VSASL signals of the blood bolus for voxels corresponding to the target area;
for each of the voxels,
obtaining signal intensity information over a cardiac cycle by performing retroactive cardiac gating on the acquired VSASL signals corresponding to the voxel; and
determining a pulsatility index for the voxel based on the signal intensity information; and
generating a voxel-wise pulsatility index map for the target area using the pulsatility indexes of the voxels.

2. The method of claim 1, wherein the application of the second VS pulse sequence is separated from the application of the first VS pulse sequence by a bolus duration.

3. The method of claim 2, further comprising selecting the bolus duration based on a cardiac period of the subject.

4. The method of claim 3, wherein the bolus duration is half of the cardiac period of the subject.

5. The method of claim 1, further comprising: selecting the cutoff velocity based on a dimension of blood vessels in the target area or a location of the target area along an arterial network of the subject.

6. The method of claim 1, wherein determining the pulsatility index for the voxel based on the signal intensity information comprises:
determining a maximum signal intensity, a minimum signal intensity, and a mean signal intensity of the signal intensity information of the voxel; and
determining the pulsatility index of the voxel based on the maximum signal intensity, the minimum signal intensity, and the mean signal intensity.

7. The method of claim 1, wherein acquiring VSASL signals of the blood bolus for the voxels comprises:
obtaining raw VSASL signals by performing a VSASL scan over the target area; and
obtaining the VSASL signals for the voxels by performing at least one of co-registration or motion correction of the raw VSASL signals.

8. The method of claim 7, wherein the VSASL scan follows the application of the second VS pulse sequence by a post-labelling delay (PLD).

9. The method of claim 8, wherein the target area is in the brain of the subject, the method further comprising: applying at least one of a spectrally-selective fat-saturation module or an inferior saturation module within the PLD.

10. The method of claim 1, wherein performing retroactive cardiac gating on VSASL signals corresponding to the voxel comprises: assigning a cardiac phase to each of the VSASL signals.

11. The method of claim 1, wherein at least one of the first VS pulse sequence or the second VS pulse sequence comprises an eight-segment B₀/B₁⁺ insensitive rotation (BIR-8) train.

12. A magnetic resonance imaging (MRI) system, comprising:
a scanner comprising a magnet;
gradient coils; and
at least one processor, wherein the at least one processor is configured to perform a process including:
magnetically labelling blood flow in a target area of a subject using a velocity-selective arterial spin labeling (VSASL) technique with a cutoff velocity by performing operations including:
applying a first velocity-selective (VS) pulse sequence with the cutoff velocity to mark a leading edge of a blood bolus; and
applying a second VS pulse sequence with the cutoff velocity to mark a trailing edge of the blood bolus;
acquiring VSASL signals of the blood bolus for voxels corresponding to the target area;
for each of the voxels,
obtaining signal intensity information over a cardiac cycle by performing retroactive cardiac gating on VSASL signals corresponding to the voxel; and
determining a pulsatility index for the voxel based on the signal intensity information; and
generating a voxel-wise pulsatility index map for the target area using the pulsatility indexes of the voxels.

13. The MRI system of claim 12, wherein a magnetic field strength of the MRI system is lower than 7 Tesla.

14. The MRI system of claim 12, wherein the application of the second VS pulse sequence is separated from the application of the first VS pulse sequence by a bolus duration.

15. The MRI system of claim 14, wherein the bolus duration relates to a cardiac period of the subject.

16. The MRI system of claim 12, wherein the cutoff velocity based on a dimension of blood vessels in the target area or a location of the target area along an arterial network of the subject.

17. The MRI system of claim 12, wherein determining the pulsatility index for the voxel based on the signal intensity information comprises:
determining a maximum signal intensity, a minimum signal intensity, and a mean signal intensity of the signal intensity information of the voxel; and
determining the pulsatility index of the voxel based on the maximum signal intensity, the minimum signal intensity, and the mean signal intensity.

18. The MRI system of claim 12, wherein the target area comprises the brain, a lung, a kidney, or the liver of the subject.

19. The MRI system of claim 12, wherein performing retroactive cardiac gating on VSASL signals corresponding to the voxel comprises: assigning a cardiac phase to each of the VSASL signals.

20. One or more computer readable media having processor-executable code, upon execution by one or more processors, causing the one or more processors to perform a process including:
magnetically labelling blood flow in a target area of a subject using a velocity-selective arterial spin labeling (VSASL) technique with a cutoff velocity by performing operations including:
applying a first velocity-selective pulse sequence with the cutoff velocity to mark a leading edge of a blood bolus; and
applying a second VS pulse sequence with the cutoff velocity to mark a trailing edge of the blood bolus;
acquiring VSASL signals of the blood bolus for voxels corresponding to the target area;
for each of the voxels,
obtaining signal intensity information over a cardiac cycle by performing retroactive cardiac gating on VSASL signals corresponding to the voxel; and
determining a pulsatility index for the voxel based on the signal intensity information; and
generating a voxel-wise pulsatility index map for the target area using the pulsatility indexes of the voxels.
